(19) United States
(12) Patent Application Publication
(10) Pub. No.: US 2021/0213037 A1
(43) Pub. Date: Jul. 15, 2021

(54) METHODS FOR TREATING FIBROSIS

(71) Applicant: CHILDREN'S HOSPITAL MEDICAL CENTER, Cincinnati, OH (US)

(72) Inventors: Satish Kumar MADALA, Blue Ash, OH (US); Anil Goud JEGGA, West Chester, OH (US); Rajesh K. KASAM, Cincinnati, OH (US)

(73) Assignee: CHILDREN'S HOSPITAL MEDICAL CENTER, Cincinnati, OH (US)

(21) Appl. No.: 16/967,441

(22) PCT Filed: Feb. 14, 2019
(86) PCT No.: PCT/US2019/017917

§ 371 Date: Aug. 5, 2020

Related U.S. Application Data
(60) Provisional application No. 62/630,866, filed on Feb. 15, 2018.

(51) Int. Cl. A61K 31/675; A61K 45/06; A61K 9/00; A61P 11/00

(52) U.S. Cl. CPC A61K 31/675; A61P 11/00; A61K 9/0073; A61K 45/06

(57) ABSTRACT

Some embodiments of the invention include methods for treating an animal for fibrosis comprising one or more administrations of one or more compositions comprising one or more AURKB (Aurora kinase B) inhibitors. Other embodiments of the methods for treating further include other fibrosis treatments. Still other embodiments of the invention include methods for treating a human for lung fibrosis or idiopathic pulmonary fibrosis, comprising administering one or more compositions comprising AZD1152 or barasertib. Additional embodiments of the invention are also discussed herein.

### CROSS-REFERENCE TO RELATED APPLICATIONS

This application claims the benefit of U.S. Provisional Application No. 62/630,866, filed Feb. 15, 2018, entitled “Repurposing Barasertib for the Treatment Pulmonary Fibrosis” which is herein incorporated by reference in its entirety.

### BACKGROUND

Fibrosis is the formation of excess fibrous connective tissue. In some instances, fibrosis results in accumulation of extracellular matrix proteins.

Several compounds are known to treat fibrosis, but do so inadequately. For example, pirfenidone and nintedanib are FDA-approved drugs for the treatment of idiopathic pulmonary fibrosis. However, pirfenidone showed no effect on respiratory symptoms. And neither pirfenidone nor nintedanib had any effect on mortality. Thus, attempts to develop a clinically effective fibrosis treatment have been unsuccessful, and there is still a need to find treatments for fibrosis.

Certain embodiments of the invention address one or more of the deficiencies described above. For example, some embodiments of the invention include methods for treating an animal for fibrosis comprising one or more administrations of one or more compositions comprising one or more AURKB (Aurora kinase B) inhibitors. Other embodiments of the methods for treating further include other fibrosis treatments. Still other embodiments of the invention include methods for treating a human for lung fibrosis or idiopathic pulmonary fibrosis, comprising administering one or more compositions comprising AZD1152 or barasertib. Additional embodiments of the invention are also discussed herein.

### SUMMARY

Some embodiments of the invention include a method for treating an animal for fibrosis, comprising one or more administrations of one or more compositions comprising one or more AURKB (Aurora kinase B) inhibitors, wherein the compositions may be the same or different if there is more than one administration. In other embodiments, at least one of the one or more AURKB inhibitors is an AURKB antagonist, an AURKB partial antagonist, an AURKB inverse agonist, an AURKB partial inverse agonist, or a combination thereof. In certain embodiments, at least one of the one or more AURKB inhibitors further inhibits one or more of AURKA (Aurora kinase A), AURKC (Aurora kinase C), JAK2 (Janus kinase 2), JAK3 (Janus kinase 3), IGF-1R (Insulin-like growth factor 1 receptor), insulin receptor, MET (Hepatocyte growth factor receptor), ALK (Anaplastic lymphoma kinase), TRKA (Tropomyosin receptor kinase A), TRKB (Tropomyosin receptor kinase B), FLT3 (fms like tyrosine kinase 3), CDK1, (Cyclin-dependent kinase 1), CDK2 (Cyclin-dependent kinase 2), KDR (Kinase insert domain receptor), or a combination thereof. In still other embodiments, at least one of the one or more AURKB inhibitors further inhibits AURKA (Aurora kinase A), AURKC (Aurora kinase C), or both. In some embodiments, at least one of the one or more AURKB inhibitors is AD6 (4-[(5-bromo-1,3-thiazol-2-yl)amino]-N-methyl-benzamide); AJI-100 (N4-(2-Chlorophenyl)-N2-(4-carbamoyl)-5-fluoropyrimidine-2,4-diamine); AJI-214 (N4-(phenyl)-N2-(4-carbamoyl)-5-fluoropyrimidine-2,4-diamine); AMG-900 (CAS number 945595-80-2; N-(4-(3-(2-aminopyrimidin-4-yl)pyridin-2-yloxy)phenyl)-4-(4-methylthiophen-2-yephthalazin-1-amine); AT9283 (CAS number 896466-04-9; 1-cyclopropyl-3-[(3Z)-3-[5-(morpholin-4-ylmethyl)benzimidazol-2-ylidene]-1,2-dihydropyrazol-4-yl]urea)); Aurora Kinase Inhibitor II (AI II) (CAS number 331770-21-9; N-[4-[(6,7-dimethoxy-4-quinazolinyl)aminolphenyl]-benzamide); AZD1152 (CAS number 722543-31-9; 2-[ethyl-[3-[4-[[5-[2-(3-fluoroanilino)-2-oxoethyl]-1H-pyrazol-3-yl]amino]quinazolin-7-yl]oxypropyl]amino]ethyl dihydrogen phosphate); Barasertib (also known as AZD1152-HQPA or AZD2811) (CAS number 722544-51-6; 3-[[7-[3-[Ethyl(2-hydroxyethyl)amino]propoxy]-4-quinazolinyl]amino]-N-(3-fluorophenyl)-1H-pyrazole-5-acetamide); BI-811283 (4-((4-(((1R,2S)-2-(isopropylcarbamoyl)cyclopentyl)amino)-5-(trifluoromethyl)pyrimidin-2-yl)amino)-N-methyl-N-(1-methylpiperidin-4-yl)benzamide); BMS-754807 (CAS number 1001350-96-4; (2S)-1-[4-[(5-Cyclopropyl-1H-pyrazol-3-yl)amino]pyrrolo[2,1-f][1,2,4]triazin-2-yl]-N-(6-fluoro-3-pyridinyl)-2-methyl-2-Pyrrolidinecarboxamide); CCT129202 (CAS number 942947-93-5; 2-(4-(6-chloro-2-(4-(dimethylamino)phenyl)-3H-imidazo[4,5-b]pyridin-7-yl)piperazin-1-yl)-N-(thiazol-2-yl)acetamide); Chiauranib (CAS number 1256349-48-0; N-(2-aminophenyl)-6-((7-methoxyquinolin-4-yl)oxy)-1-naphthamide); CYC116 (CAS number 693228-63-6; 4-methyl-5-(2-(4-morpholinophenylamino)pyrimidin-4-yl)thiazol-2-amine); ENMD-2076 (CAS number 934353-76-1; (E)-N-(5-methyl-1H-pyrazol-3-yl)-6-(4-methylpiperazin-1-yl)-2-styrylpyrimidin-4-amine); GSK-1070916 (CAS number 942918-07-2; 3-(4-(4-(2-(3-((dimethylamino)methyl)phenyl)-1H-pyrrolo[2,3-b]pyridin-4-yl)-1-ethyl-1H-pyrazol-3-yl)phenyl)-1,1-dimethylurea); Hesperadin (CAS number 422513-13-1; N-[(3Z)-2-Oxo-3-[phenyl-[4-(piperidin-1-ylmethyl)anilino]methylidene]-1H-indol-5-yl]ethanesulfonamide); Ilorasertib (aka ABT-348; CAS number 1227939-82-3; 1-(4-(4-amino-7-(1-(2-hydroxyethyl)-1H-pyrazol-4-yl)thieno[3,2-c]pyridin-3-yl)phenyl)-3-(3-fluorophenyl)urea); JNJ-7706621 (CAS number 443797-96-4; 4-[5-amino-1-(2,6-difluoro-benzoyl)-1H-[1,2,4]triazol-3-ylamino]-benzenesulfonamide); KW-2449 (CAS number 1000669-72-6; (E)-(4-(2-(1H-indazol-3-yl)vinyl)phenyl)(piperazin-1-yl)methanone); KW-2450 (CAS number 904899-25-8; (E)-N-(2-(2-(1H-indazol-3-yl)vinyl)-5-((4-(2-hydroxyacetyl)piperazin-1-yl)methyl)phenyl)-3-methylthiophene-2-carboxamide 4-methylbenzenesulfonate) or its tosylate salt; MK-6592 (aka VX667; (S)-(5-chloro-2-fluorophenyl)(3-(4-(3-cyclopropyl-3-fluoroazetidin-1-yl)-6-(3-methyl-1H-pyrazol-5-ylamino)pyrimidin-2-yloxy)pyrrolidin-1-yl)methanone); MLN8054 (CAS number 869363-13-3; 4-((9-chloro-7-(2,6-difluorophenyl)-5H-benzo[c]pyrimido[4,5-e]azepin-2-yl)amino)benzoic acid); MLN8237 (CAS number 1028486-01-2; 4-{[9-Chloro-7-(2-fluoro-6-methoxyphenyl)-5H-pyrimido[5,4-d][2]benzazepin-2-yl]amino}-2-methoxybenzoic acid); PF-03814735 (CAS number 942487-16-3; N-(2-[(1S,4R)-6-[[4-(Cyclobutylamino)-5-(trifluoromethyl)-2-pyrimidinyl]amino]-1,2,3,4-tetrahydronaphthalen-1,4-imin-9-yl]-2-oxoethyl]-acetamide) or its mesylate salt; PHA-680632 (CAS number 398493-79-3; N-(2,6-diethylphenyl)-3-[[4-(4-methylpiperazin-1-yl)benzoyl]amino]-4,6-dihydro-1H-pyrrolo[3,4-c]pyrazole-5-carboxamide); PHA-739358 (aka Danusertib; CAS number 827318-97-8; 4-(4-methyl-1-piperazinyl)-N-[1,4,5,6-tetrahydro-5-[(2R)-2-methoxy-2-phenylacetyl]pyrrolo[3,4-c]pyrazol-3-yl]-benzamide); SNS314 (CAS number 1146618-41-8; 1-(3-chlorophenyl)-3-[5-[2-(thieno[3,2-d]pyrimidin-4-ylamino)ethyl]-1,3-thiazol-2-yl]urea) or its mesylate salt; SU6668 (CAS number 252916-29-3; 5-[1,2-Dihydro-2-oxo-3H-indol-3-ylidene)methyl]-2,4-dimethyl-1H-pyrrole-3-propanoic acid); TAK-901 (CAS number 934541-31-8; 5-(3-(ethylsulfonyl)phenyl)-3,8-dimethyl-N-(1-methylpiperidin-4-yl)-9H-pyrido[2,3-b]indole-7-carboxamide); TX47 (3,3′-((1H-indole-2,3-diyl)bis(methylene))bis(1H-indole)); TY-011 (9-(2-chloro-phenyl)-6-ethyl-1-methyl-2,4-dihydro-2,3,4,7,10-pentaaza-benzo[f]azulene); VX-680 (aka Tozasertib; CAS number 639089-54-6; N-[4-[4-(4-Methylpiperazin-1-yl)-6-[(5-methyl-1H-pyrazol-3-yl)amino]pyrimidin-2-yl]sulfanylphenyl]cyclopropanecarboxamide); ZM447439 (CAS number 331771-20-1; N-[4-[[6-methoxy-7-[3-(4-morpholinyl)propoxy]-4-quinazolinyl]amino]phenyl]-benzamide); or a salt, ester, or solvate of any of the aforementioned. In still other embodiments, at least one of the one or more AURKB inhibitors is AMG-900; AT-9283; AZD1152; Barasertib; BI-811283; Chiauranib; CYC-116; ENMD-2076; GSK-1070916; Ilorasertib; KW-2449; MK-6592; PF-03814735 or its mesylate salt; PHA-739358 (aka Danusertib); TAK-901; SNS-314 or its mesylate salt; VX-680 (aka Tozasertib); or a salt, ester, or solvate of any of the aforementioned. In still other embodiments, at least one of the one or more AURKB inhibitors is AD6; AJI-100; AJI-214; AT9283; Aurora Kinase Inhibitor II (AI II); AZD1152; Barasertib (aka AZD1152-HQPA); BMS-754807; CYC116; hesperadin; JNJ-7706621; KW-2450 or its tosylate salt; PF-03814735 or its mesylate salt; TX47; TY-011; ZM447439; or a salt, ester, or solvate of any of the aforementioned. In yet other embodiments, at least one of the one or more AURKB inhibitors is BRD-7880, barasertib, AZD1152, GSK1070916, TAK-901 or a salt, ester, or solvate of any of the aforementioned. In certain embodiments, at least one of the one or more AURKB inhibitors is barasertib or AZD1152, or a salt, ester, or solvate of barasertib or of AZD1152. In other embodiments, at least one of the one or more AURKB inhibitors is barasertib or AZD1152.

In some embodiments, the amount of at least one of the one or more AURKB inhibitors is from about 0.0001% (by weight total composition) to about 99%. In other embodiments, at least one of the one or more compositions further comprises a formulary ingredient. In certain embodiments, at least one of the one or more compositions is a pharmaceutical composition.

In other embodiments, at least one of the one or more administrations comprises a parenteral administration, a mucosal administration, an intravenous administration, a depot injection, a subcutaneous administration, a topical administration, an intradermal administration, an oral administration, a sublingual administration, an intratracheal administration, an intranasal administration, an intramuscular administration, an aerosol administration, a nebulizer administration, a pressurized metered-dose inhaler (pMDI) administration, an inhaler administration, or a dry powder inhaler (DPI) administration. In still other embodiments, at least one of the one or more administrations comprises an intratracheal administration, an intranasal administration, an aerosol administration, a nebulizer administration, a pressurized metered-dose inhaler (pMDI) administration, an inhaler administration, or a dry powder inhaler (DPI) administration. In yet other embodiments, if there is more than one administration at least one composition used for at least one administration is different from the composition of at least one other administration.

In other embodiments, at least one AURKB inhibitor of at least one of the one or more compositions is administered to the animal in an amount of from about 0.005 mg/kg animal body weight to about 100 mg /kg animal body weight. In yet other embodiments, the animal is a human, a rodent, or a primate. In still other embodiments, the animal is in need of treatment of fibrosis.

In some embodiments, the method is for treating lung fibrosis, skin fibrosis, kidney fibrosis, liver fibrosis, gastrointestinal fibrosis, heart fibrosis, brain fibrosis, arterial stiffness, arthrofibrosis, crohn's disease, dupuytren's contracture, keloid, mediastinal fibrosis, myelofibrosis, peyronie's disease, nephrogenic systemic fibrosis, progressive massive fibrosis, retroperitoneal fibrosis, scleroderma/systemic sclerosis, adhesive capsulitis, or other organ fibrosis. In other embodiments, the method is for treating lung fibrosis, pulmonary fibrosis, cystic fibrosis, idiopathic pulmonary fibrosis (IPF), radiation-induced lung injury, skin fibrosis, kidney fibrosis, liver fibrosis, cirrhosis, heart fibrosis, atrial fibrosis, endomyocardial fibrosis, myocardial infarction, gastrointestinal fibrosis, fibrosis of the gastrointestinal tract, fibrosis associated with gastrointestinal inflammation, fibrosis associated with inflammatory bowel disease, fibrosis associated with ulcerative colitis, fibrosis associated with Crohn's disease, intestine fibrosis, small intestine fibrosis, ilium fibrosis, cecum fibrosis, or colon fibrosis. In still other embodiments, the method is for treating lung fibrosis, pulmonary fibrosis, cystic fibrosis, idiopathic pulmonary fibrosis (IPF), or radiation-induced lung injury. In yet other embodiments, the method is for treating gastrointestinal fibrosis, fibrosis of the gastrointestinal tract, fibrosis associated with gastrointestinal inflammation, fibrosis associated with inflammatory bowel disease, fibrosis associated with ulcerative colitis, fibrosis associated with Crohn's disease, intestine fibrosis, small intestine fibrosis, ilium fibrosis, cecum fibrosis, or colon fibrosis. In certain embodiments, the method is for treating liver fibrosis, kidney fibrosis, or skin fibrosis.

In some embodiments, the method further comprises one or more other fibrosis treatments. In other embodiments, the method further comprises one or more other fibrosis treatments and the other fibrosis treatment comprises administering one or more of an antibiotic, an anti-inflammatory drug, a mucus thinner, or an antifibrotic medication. In still other embodiments, the method further comprises one or more other fibrosis treatments and the other fibrosis treatment comprises administering one or more non-drug respiratory therapies.

Some embodiments of the invention include a method for treating a human for lung fibrosis, pulmonary fibrosis, or idiopathic pulmonary fibrosis (IPF), comprising administering one or more compositions comprising barasertib or AZD1152. In other embodiments, at least one of the one or more compositions comprises AZD1152.

Some embodiments of the invention include a method for treating a human for idiopathic pulmonary fibrosis (IPF), comprising administering one or more compositions comprising barasertib or AZD1152, wherein the administering is by a pressurized metered-dose inhaler (pMDI) administration, an inhaler administration, or a dry powder inhaler (DPI) administration. In other embodiments, at least one of the one or more compositions comprises barasertib.

Other embodiments of the invention are also discussed herein.

### BRIEF DESCRIPTION OF THE DRAWINGS

The following drawings form part of the present specification and are included to further demonstrate certain aspects of the present invention. The invention may be better understood by reference to one or more of these drawings in combination with the description of specific embodiments presented herein.

FIG. 1: Network representation of barasertib regulated genes in IPF. The biological function enrichment analysis of genes that are upregulated in IPF but downregulated by barasertib (orange) and vice versa (violet).

FIG. 2: AURKB is upregulated in mesenchymal cells that accumulate in IPF lungs. IPF and Normal lung sections were immunostained with antibody against AURK-B (brown). AURKB was localized in nuclear regions of spindle shaped lung mesenchymal cells (arrows).

FIG. 3: AURKB is upregulated in IPF subtypes and correlated with the severity of lung function decline and fibrosis. Expression of AURKB compared with lung function parameters in controls and patients with IPF. A clear negative correlation is evident between AURKB and lung function parameters (1) FVC (forced vital capacity, which is the maximum amount of air exhaled after a maximal inhalation and (2) DLCO (aka transfer factor for carbon monoxide, which is used to measure the ability of the lungs to transfer gas from inhaled air to the red blood cells in pulmonary capillaries) (r²=0.103 and 0.089, respectively).

FIG. 4: TGFα induces AURKB expression lung resident fibrobalsts. AURKB transcripts were quantified using RT-PCR in fibroblasts isolated from human lungs and treated with TGFα, TGFβ, CTGF and IGF1 (50 ng/ml). **p<0.005; ***p<0.0005; ****p<0.00005.

FIG. 5: Comparison of pathologic features on an H&E stained lung biopsy from a patient with IPF (top) and from TGFα transgenic mice (bottom). In the presence of doxycycline (Dox), the activator transgene (CCSP-rtTA) is activated and binds to the tetO-promoter (tetO-TGFα), which causes overexpression of TGFα by airway epithelial cells. TGFα mice develop fibrotic lesions in the lung subpleura and parenchyma with histological features similar to IPF.

FIG. 6: AURKB is upregulated during TGFα-induced in pulmonary fibrosis. Immunoblots show AURK-B but not AURK-A increase in the lung lysates of TGFα mice compared to control mice on Dox for 4 wks.

FIG. 7: AURKB is upregulated during bleomycin-induced in pulmonary fibrosis. AURK-B upregulated in the lungs of bleomycin-treated mice for 4 wks compared to saline treated control mice.

FIG. 8: AURKB is a positive regulator of fibroproliferation. (A) Proliferation was measured using BrdU incorporation assay in primary fibroblasts isolated from the lungs of TGFα mice on Dox for 4 wks, and treated with control or AURKB-specific siRNA for 72 hr. (B) Extent of proliferation was measured in IPF lung fibroblasts treated with control or AURKB-specific siRNA for 72 hr. (C) Quantitation of proliferation using the Brdu incorporation assay in IPF lung fibroblasts treated with indicated doses of barasertib or vehicle for 48 hr. ****p<0.00005.

FIG. 9: AURKB is upregulated in myofibroblasts. AURK-B upregulated in myofibroblasts localized in the mature fibrotic lung lesions of TGFα mice on Dox for 6 wks compared to saline treated control mice.

FIG. 10: The loss of AURK-B attenuates fibroblast survival. (A) Fibroblasts of TGFα mice on Dox for 6 wks were treated with control or AURKB-specific siRNA for 48 hrs. FasL-induced apoptosis was analyzed using Incucyte. (B) IPF fibroblasts were treated with control or AURKB-specific siRNA for 48 hrs., and FasL-induced apoptosis was analyzed using Incucyte. **P<0.005; ****P<0.00005.

FIG. 11: Barasertib therapy attenuates pulmonary fibrosis in vivo. Mice were treated intraperitoneally (i.p.) with either vehicle or Barasertib (40 mg/kg bodyweight, QD) for 4 wks on Dox. (A) Masson Trichrome staining shows attenuation of collagen deposition in subpleural fibrotic lesions of TGFα mice treated with barasertib compared to vehicle. (B) Total lung hydroxyproline levels were attenuated in mice treated with barasertib. *P<0.05; **P<0.005.

FIG. 12: Therapeutic intervention with barasertib attenuates fibroproliferation. All groups of mice on Dox for two weeks were treated intraperitoneally (i.p.) with either vehicle, barasertib (25 mg/kg or 50 mg/kg, BID) or nintedanib (60 mg/kg, QD). Total lung RNA was analyzed for the expression of proliferative genes, Plk1 and IL-6 using RT-PCR. *P<0.05; **P<0.005.

FIG. 13: Therapeutic intervention with barasertib attenuates fibroblast survival gene expression. All groups of mice on Dox for two weeks were treated intraperitoneally (i.p.) with either vehicle, barasertib (25 mg/kg or 50 mg/kg, QD) or nintedanib (60 mg/kg, QD). Total lung RNA was analyzed for the expression of pro-apoptotic genes, Bak1 and Fas using RT-PCR. *P<0.05; **P<0.005.

FIG. 14: Barasertib attenuates ECM gene expression. All groups of mice on Dox for two weeks were treated intraperitoneally (i.p.) with either vehicle, barasertib (25 mg/kg or 50 mg/kg bodyweight, QD) or nintedanib (60 mg/kg, QD). Total lung RNA was analyzed for the expression of ECM genes, Co11α and FN1 using RT-PCR. *P<0.05; **P<0.005.

### DETAILED DESCRIPTION

While embodiments encompassing the general inventive concepts may take diverse forms, various embodiments will be described herein, with the understanding that the present disclosure is to be considered merely exemplary, and the general inventive concepts are not intended to be limited to the disclosed embodiments.

Some embodiments of the invention include methods for treating an animal for fibrosis comprising one or more administrations of one or more compositions comprising one or more AURKB (Aurora kinase B) inhibitors. Other embodiments of the methods for treating further include other fibrosis treatments. Still other embodiments of the invention include methods for treating a human for lung fibrosis or idiopathic pulmonary fibrosis, comprising administering one or more compositions comprising AZD1152 or barasertib. Additional embodiments of the invention are also discussed herein.

### Treatments of Disease

Some embodiments of the invention include treatment of disease (e.g., fibrosis) by administering one or more Aurora kinase B (AURKB) inhibitors. One or more AURKB inhibitors (e.g., barasertib or AZD1152) can be administered to animals by any number of suitable administration routes or formulations. One or more AURKB inhibitors (e.g., barasertib or AZD1152) can also be used to treat animals for a variety of diseases. Animals include but are not limited to mammals, primates, monkeys (e.g., macaque, rhesus macaque, or pig tail macaque), humans, canine, feline, bovine, porcine, avian (e.g., chicken), mice, rabbits, and rats. As used herein, the term “subject” refers to both human and animal subjects.

The route of administration of one or more AURKB inhibitors (e.g., barasertib or AZD1152) can be of any suitable route. Administration routes can be, but are not limited to the oral route, the parenteral route, the cutaneous route, the nasal route, the rectal route, the vaginal route, and the ocular route. In other embodiments, administration routes can be parenteral administration, a mucosal administration, intravenous administration, depot injection, subcutaneous administration, topical administration, intradermal administration, oral administration, sublingual administration, intratracheal administration, intranasal administration, or intramuscular administration. In some embodiments, the administration can be an intratracheal administration, intranasal administration, an aerosol administration, a nebulizer administration, a pressurized metered-dose inhaler (pMDI) administration, an inhaler administration, or a dry powder inhaler (DPI) administration. The choice of administration route can depend on the compound identity (e.g., the physical and chemical properties of the compound) as well as the age and weight of the animal, the particular disease (e.g., fibrosis), and the severity of the disease (e.g., stage or severity of disease). Of course, combinations of administration routes can be administered, as desired.

Some embodiments of the invention include a method for providing a subject with a composition comprising one or more AURKB inhibitors (e.g., barasertib or AZD1152) described herein (e.g., a pharmaceutical composition) which comprises one or more administrations of one or more such compositions; the compositions may be the same or different if there is more than one administration.

Diseases that can be treated in an animal (e.g., mammals, porcine, canine, avian (e.g., chicken), bovine, feline, primates, rodents, monkeys, rabbits, mice, rats, and humans) using one or more AURKB inhibitors include, but are not limited to fibrosis.

In some embodiments, fibrosis that can be treated in an animal (e.g., mammals, porcine, canine, avian (e.g., chicken), bovine, feline, primates, rodents, monkeys, rabbits, mice, rats, and humans) using an AURKB inhibitor include, but are not limited to lung fibrosis, pulmonary fibrosis, cystic fibrosis, idiopathic pulmonary fibrosis (IPF), or radiation-induced lung injury. In some embodiments, fibrosis that can be treated include, but are not limited to, lung fibrosis (e.g., pulmonary fibrosis, cystic fibrosis, idiopathic pulmonary fibrosis (IPF), radiation-induced lung injury, or radiation-induced lung injury resulting from treatment for cancer), skin fibrosis, kidney fibrosis, liver fibrosis (e.g., cirrhosis), gastrointestinal fibrosis (e.g., fibrosis of the gastrointestinal tract, fibrosis associated with gastrointestinal inflammation, fibrosis associated with inflammatory bowel disease, fibrosis associated with ulcerative colitis, fibrosis associated with Crohn's disease, intestine fibrosis, small intestine fibrosis, ilium fibrosis, cecum fibrosis, or colon fibrosis), heart fibrosis (e.g., atrial fibrosis, endomyocardial fibrosis, or myocardial infarction), brain fibrosis (e.g., glial scar), or other forms of fibrosis including but not limited to arterial stiffness, arthrofibrosis (e.g., knee, shoulder, or other joints), crohn's disease (e.g., intestine), dupuytren's contracture (e.g., hand or finger), keloid (e.g., skin), mediastinal fibrosis (e.g., soft tissue of the mediastinum), myelofibrosis (e.g., bone marrow), peyronie's disease (e.g., penis), nephrogenic systemic fibrosis (e.g., skin), progressive massive fibrosis (e.g., a complication of coal workers' pneumoconiosis), retroperitoneal fibrosis (e.g., soft tissue of the retroperitoneum), scleroderma/systemic sclerosis (e.g., skin or lung), adhesive capsulitis (e.g., shoulder), or other organ fibrosis. In other embodiments, fibrosis that can be treated can include lung fibrosis, kidney fibrosis, skin fibrosis, liver fibrosis, heart fibrosis, brain fibrosis, or gastrointestinal fibrosis. In other embodiments, fibrosis that can be treated can include lung fibrosis, kidney fibrosis, liver fibrosis, heart fibrosis, skin fibrosis, or gastrointestinal fibrosis. In other embodiments, fibrosis that can be treated can include lung fibrosis, liver fibrosis, heart fibrosis, or gastrointestinal fibrosis. In certain embodiments, fibrosis that can be treated can include lung fibrosis, pulmonary fibrosis, cystic fibrosis, idiopathic pulmonary fibrosis (IPF), radiation-induced lung injury, liver fibrosis, cirrhosis, heart fibrosis, atrial fibrosis, endomyocardial fibrosis, myocardial infarction, gastrointestinal fibrosis, fibrosis of the gastrointestinal tract, fibrosis associated with gastrointestinal inflammation, fibrosis associated with inflammatory bowel disease, fibrosis associated with ulcerative colitis, fibrosis associated with Crohn's disease, intestine fibrosis, small intestine fibrosis, ilium fibrosis, cecum fibrosis, or colon fibrosis. In certain embodiments, fibrosis that can be treated can include lung fibrosis, pulmonary fibrosis, cystic fibrosis, idiopathic pulmonary fibrosis (IPF), radiation-induced lung injury, skin fibrosis, kidney fibrosis, liver fibrosis, cirrhosis, heart fibrosis, atrial fibrosis, endomyocardial fibrosis, or myocardial infarction. In certain embodiments, fibrosis that can be treated can include lung fibrosis, pulmonary fibrosis, cystic fibrosis, idiopathic pulmonary fibrosis (IPF), radiation-induced lung injury, kidney fibrosis, liver fibrosis, cirrhosis, heart fibrosis, atrial fibrosis, endomyocardial fibrosis, or myocardial infarction. In other embodiments, fibrosis that can be treated can include lung fibrosis, pulmonary fibrosis, cystic fibrosis, idiopathic pulmonary fibrosis (IPF), radiation-induced lung injury, skin fibrosis, kidney fibrosis, heart fibrosis, atrial fibrosis, endomyocardial fibrosis, or myocardial infarction. In other embodiments, fibrosis that can be treated can include lung fibrosis, pulmonary fibrosis, cystic fibrosis, idiopathic pulmonary fibrosis (IPF), radiation-induced lung injury, kidney fibrosis, heart fibrosis, atrial fibrosis, endomyocardial fibrosis, or myocardial infarction. In other embodiments, fibrosis that can be treated can include lung fibrosis, pulmonary fibrosis, cystic fibrosis, idiopathic pulmonary fibrosis (IPF), radiation-induced lung injury, heart fibrosis, atrial fibrosis, endomyocardial fibrosis, or myocardial infarction. In other embodiments, fibrosis that can be treated can include lung fibrosis, pulmonary fibrosis, cystic fibrosis, idiopathic pulmonary fibrosis (IPF), or radiation-induced lung injury.

Animals that can be treated include but are not limited to mammals, rodents, primates, monkeys (e.g., macaque, rhesus macaque, pig tail macaque), humans, canine, feline, porcine, avian (e.g., chicken), bovine, mice, rabbits, and rats. As used herein, the term “subject” refers to both human and animal subjects. In some instances, the animal is in need of the treatment (e.g., by showing signs of disease or fibrosis).

In some embodiments, fibrosis that can be treated in an animal (e.g., mammals, porcine, canine, avian (e.g., chicken), bovine, feline, primates, rodents, monkeys, rabbits, mice, rats, and humans) using one or more AURKB inhibitors include, but are not limited to fibrosis that can be treated by inhibiting (e.g., reducing the activity or expression of) AURKB.

As used herein, the term “treating” (and its variations, such as “treatment”) is to be considered in its broadest context. In particular, the term “treating” does not necessarily imply that an animal is treated until total recovery. Accordingly, “treating” includes amelioration of the symptoms, relief from the symptoms or effects associated with a condition, decrease in severity of a condition, or preventing, preventively ameliorating symptoms, or otherwise reducing the risk of developing a particular condition. As used herein, reference to “treating” an animal includes but is not limited to prophylactic treatment and therapeutic treatment. Any of the compositions (e.g., pharmaceutical compositions) described herein can be used to treat an animal.

As related to treating fibrosis (e.g., lung fibrosis, pulmonary fibrosis, cystic fibrosis, idiopathic pulmonary fibrosis (IPF), or radiation-induced lung injury), treating can include but is not limited to prophylactic treatment and therapeutic treatment. As such, treatment can include, but is not limited to: preventing fibrosis (e.g., lung fibrosis, pulmonary fibrosis, cystic fibrosis, idiopathic pulmonary fibrosis (IPF), or radiation-induced lung injury); reducing the risk of fibrosis (e.g., lung fibrosis, pulmonary fibrosis, cystic fibrosis, idiopathic pulmonary fibrosis (IPF), or radiation-induced lung injury); ameliorating or relieving symptoms of fibrosis (e.g., lung fibrosis, pulmonary fibrosis, cystic fibrosis, idiopathic pulmonary fibrosis (IPF), or radiation-induced lung injury); eliciting a bodily response against fibrosis (e.g., lung fibrosis, pulmonary fibrosis, cystic fibrosis, idiopathic pulmonary fibrosis (IPF), or radiation-induced lung injury); inhibiting the development or progression of fibrosis (e.g., lung fibrosis, pulmonary fibrosis, cystic fibrosis, idiopathic pulmonary fibrosis (IPF), or radiation-induced lung injury); inhibiting or preventing the onset of symptoms associated with fibrosis (e.g., lung fibrosis, pulmonary fibrosis, cystic fibrosis, idiopathic pulmonary fibrosis (IPF), or radiation-induced lung injury); reducing the severity of fibrosis (e.g., lung fibrosis, pulmonary fibrosis, cystic fibrosis, idiopathic pulmonary fibrosis (IPF), or radiation-induced lung injury); causing a regression of fibrosis (e.g., lung fibrosis, pulmonary fibrosis, cystic fibrosis, idiopathic pulmonary fibrosis (IPF), or radiation-induced lung injury) or one or more of the symptoms associated with fibrosis (e.g., a decrease in the amount of fibrosis); causing remission of fibrosis (e.g., lung fibrosis, pulmonary fibrosis, cystic fibrosis, idiopathic pulmonary fibrosis (IPF), or radiation-induced lung injury); or preventing relapse of fibrosis (e.g., lung fibrosis, pulmonary fibrosis, cystic fibrosis, idiopathic pulmonary fibrosis (IPF), or radiation-induced lung injury). In some embodiments, treating does not include prophylactic treatment of fibrosis (e.g., preventing or ameliorating future fibrosis).

Treatment of an animal (e.g., human) can occur using any suitable administration method (such as those disclosed herein) and using any suitable amount of a compound of an AURKB inhibitor (e.g., barasertib or AZD1152). In some embodiments, methods of treatment comprise treating an animal for fibrosis (e.g., lung fibrosis, pulmonary fibrosis, cystic fibrosis, idiopathic pulmonary fibrosis (IPF), or radiation-induced lung injury). Some embodiments of the invention include a method for treating a subject (e.g., an animal such as a human or primate) with a composition comprising one or more AURKB inhibitors (e.g., barasertib or AZD1152) (e.g., a pharmaceutical composition) which comprises one or more administrations of one or more such compositions; the compositions may be the same or different if there is more than one administration.

In some embodiments, the method of treatment includes administering an effective amount of a composition comprising one or more AURKB inhibitors (e.g., barasertib or AZD1152). As used herein, the term “effective amount” refers to a dosage or a series of dosages sufficient to affect treatment (e.g., to treat fibrosis, such as but not limited to lung fibrosis, pulmonary fibrosis, cystic fibrosis, idiopathic pulmonary fibrosis (IPF), or radiation-induced lung injury) in an animal. In some embodiments, an effective amount can encompass a therapeutically effective amount, as disclosed herein. In certain embodiments, an effective amount can vary depending on the subject and the particular treatment being affected. The exact amount that is required can, for example, vary from subject to subject, depending on the age and general condition of the subject, the particular adjuvant being used (if applicable), administration protocol, and the like. As such, the effective amount can, for example, vary based on the particular circumstances, and an appropriate effective amount can be determined in a particular case. An effective amount can, for example, include any dosage or composition amount disclosed herein. In some embodiments, an effective amount of one or more AURKB inhibitors (for example, but not limited to barasertib or AZD1152) (which can be administered to an animal such as mammals, primates, monkeys or humans) can be an amount of about 0.005 to about 50 mg/kg body weight, about 0.005 to about 80 mg/kg body weight, about 0.005 to about 100 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 0.5 mg/kg, about 1 mg/kg, about 3 mg/kg, about 5 mg/kg, about 5.5 mg/kg, about 6 mg/kg, about 6.5 mg/kg, about 7 mg/kg, about 7.5 mg/kg, about 8 mg/kg, about 10 mg/kg, about 12 mg/kg, or about 15 mg/kg. In regard to some embodiments, the dosage can be about 0.5 mg/kg human body weight, about 5 mg/kg human body weight, about 6.5 mg/kg human body weight, about 10 mg/kg human body weight, about 50 mg/kg human body weight, about 80 mg/kg human body weight, or about 100 mg/kg human body weight. In some instances, an effective amount of one or more AURKB inhibitors (for example, but not limited to barasertib or AZD1152) (which can be administered to an animal such as mammals, rodents, mice, rabbits, feline, porcine, or canine) can be an amount of about 0.005 to about 50 mg/kg body weight, about 0.005 to about 100 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 1 mg/kg, about 5 mg/kg, about 10 mg/kg, about 20 mg/kg, about 30 mg/kg, about 40 mg/kg, about 50 mg/kg, about 80 mg/kg, about 100 mg/kg, or about 150 mg/kg. In some embodiments, an effective amount of one or more AURKB inhibitors (for example, but not limited to barasertib or AZD1152) (which can be administered to an animal such as mammals, primates, monkeys or humans) can be an amount of about 1 to about 1000 mg/kg body weight, about 5 to about 500 mg/kg body weight, about 10 to about 200 mg/kg body weight, about 25 to about 100 mg/kg body weight, about 1 mg/kg, about 2 mg/kg, about 5 mg/kg, about 10 mg/kg, about 25 mg/kg, about 50 mg/kg, about 100 mg/kg, about 150 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1000 mg/kg. In regard to some conditions, the dosage can be about 5 mg/kg human body weight, about 10 mg/kg human body weight, about 20 mg/kg human body weight, about 80 mg/kg human body weight, or about 100 mg/kg human body weight. In some instances, an effective amount of one or more AURKB inhibitors (for example, but not limited to barasertib or AZD1152) (which can be administered to an animal such as mammals, rodents, mice, rabbits, feline, porcine, or canine) can be an amount of about 1 to about 1000 mg/kg body weight, about 5 to about 500 mg/kg body weight, about 10 to about 200 mg/kg body weight, about 25 to about 100 mg/kg body weight, about 1 mg/kg, about 2 mg/kg, about 5 mg/kg, about 10 mg/kg, about 25 mg/kg, about 50 mg/kg, about 80 mg/kg, about 100 mg/kg, about 150 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1000 mg/kg.

“Therapeutically effective amount” means an amount effective to achieve a desired and/or beneficial effect (e.g., decreasing amount of fibrosis). A therapeutically effective amount can be administered in one or more administrations. For some purposes of this invention, a therapeutically effective amount is an amount appropriate to treat an indication (e.g., to treat fibrosis). By treating an indication is meant achieving any desirable effect, such as one or more of palliate, ameliorate, stabilize, reverse, slow, or delay disease (e.g., fibrosis) progression, increase the quality of life, or to prolong life. Such achievement can be measured by any suitable method, such as but not limited to measurement of the amount of fibrosis, the number of fibrocytes, the number of fibroblasts, the number of myofibroblasts, the extent of subpleural lung thickening, lung weight, body weight, lung function, or any suitable method to assess the progression of pulmonary fibrosis.

In some embodiments, other fibrosis treatments are optionally included, and can be used with the inventive treatments described herein (e.g., administering AURKB inhibitors). Other fibrosis treatments can include any known fibrosis treatment that is suitable to treat fibrosis. Examples of known fibrosis treatments include but are not limited to administration of: antibiotics (e.g., penicillins, methicillin, oxacillin, nafcillin, cabenicillin, ticarcillin, piperacillin, mezlocillin, azlocillin, ticarcillin clavulanic acid, piperacillin tazobactam, cephalosporins, cephalexin, cefdinir, cefprozil, cefaclor, cefepime, sulfa, sulfamethoxazole, trimethoprim, erythromycin/sulfisoxazole, macrolides, erythromycin, clarithromycin, azithromycin, tetracyclines, tetracycline, doxycycline, minocycline, tigecycline, vancomycin, imipenem, meripenem, colistimethate/colistin, aminoglycosides, tobramycin, amikacin, gentamicin, quinolones, aztreonam, or linezolid), anti-inflammatory drugs (e.g., NSAIDs, aspirin, ibuprofen, naproxen, corticosteroids, cortisol, corticosterone, cortisone, or aldosterone), bronchodilators (e.g., albuterol or levalbuterol hydrochloride), mucus thinners (e.g., hypertonic saline or Dornase alfa), and antifibrotic medications (e.g., pirfenidone, nintedanib, N-acetylcysteine, ivacaftor, or lumacaftor/ivacaftor). Other fibrosis treatment can also include administering a non-drug respiratory therapy such as but not limited to airway clearance techniques (e.g., postural drainage and chest percussion, exercise, breathing exercises, or use of mechanical equipment such as high-frequency chest compression vest or positive expiratory pressure therapy). Other fibrosis treatment can also include organ transplantation (e.g., lung, skin, kidney, liver, heart, small intestine, or colon).

In some embodiments, administration of an opioid receptor inhibitor, naltrexone, pirfenidone, nintedanib, or a combination thereof can be used as part of the treatment regime (i.e., as an other fibrosis treatment, in addition to administration of one or more AURKB inhibitors); administration of an opioid receptor inhibitor, naltrexone, pirfenidone, nintedanib, or a combination thereof, can include separate administrations (i.e., in a separate composition from the AURKB inhibitor) or can be added to the composition comprising the AURKB inhibitor.

In some embodiments, additional optional treatments (e.g., as an other fibrosis treatment) can also include one or more of surgical intervention, hormone therapies, immunotherapy, and adjuvant systematic therapies.

### AURKB Inhibitors

In some embodiments of the invention, any suitable AURKB can be used in the methods described herein, including but not limited methods for treating fibrosis (e.g., lung fibrosis, pulmonary fibrosis, cystic fibrosis, idiopathic pulmonary fibrosis (IPF), radiation-induced lung injury, or radiation-induced lung injury resulting from treatment for cancer).

In some embodiments, AURKB inhibitors can inhibit (e.g., fully inhibit or partially inhibit) one or more AURKBs by, for example, reducing the activity or expression of an AURKB. In other embodiments, AURKB inhibitors can be AURKB antagonists, AURKB partial antagonists, AURKB inverse agonists, AURKB partial inverse agonists, or combinations thereof. In certain embodiments, inhibition can occur using any suitable mechanism, such as but not limited to blockading the receptor (e.g., partially or fully blocking other molecules from accessing one or more receptor sites), an antagonist mechanism, a partial antagonist mechanism, an inverse agonist mechanism, a partial inverse agonist mechanism, or a combination thereof.

In some embodiments, AURKBs that can be inhibited include any suitable AURKB that can be inhibited to treat fibrosis. In other embodiments, the AURKB inhibitor can, in some embodiments, inhibit one or more of the following: AURKA (Aurora kinase A), AURKC (Aurora kinase C), JAK2 (Janus kinase 2), JAK3 (Janus kinase 3), IGF-1R (Insulin-like growth factor 1 receptor), insulin receptor, MET (Hepatocyte growth factor receptor), ALK (Anaplastic lymphoma kinase), TRKA (Tropomyosin receptor kinase A), TRKB (Tropomyosin receptor kinase B), FLT3 (fms like tyrosine kinase 3), CDK1, (Cyclin-dependent kinase 1), CDK2 (Cyclin-dependent kinase 2), or KDR (Kinase insert domain receptor).

In some embodiments, the AURKB inhibitor can include any suitable AURKB inhibitor to treat fibrosis (e.g., lung fibrosis, pulmonary fibrosis, cystic fibrosis, or idiopathic pulmonary fibrosis (IPF)). In other embodiments, the AURKB inhibitor can be, but is not limited to, an AURKB antagonist, an AURKB partial antagonist, an AURKB inverse agonist, or an AURKB partial inverse agonist, or a combination thereof.

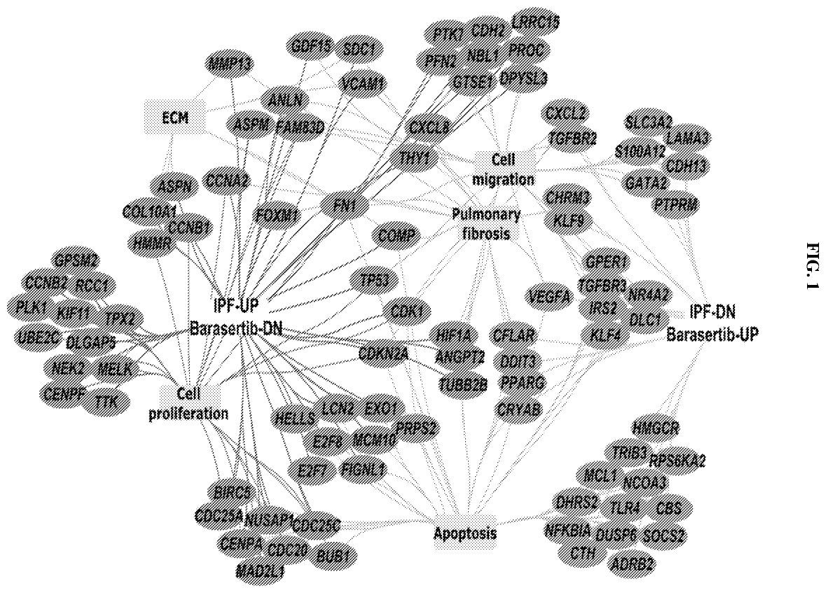

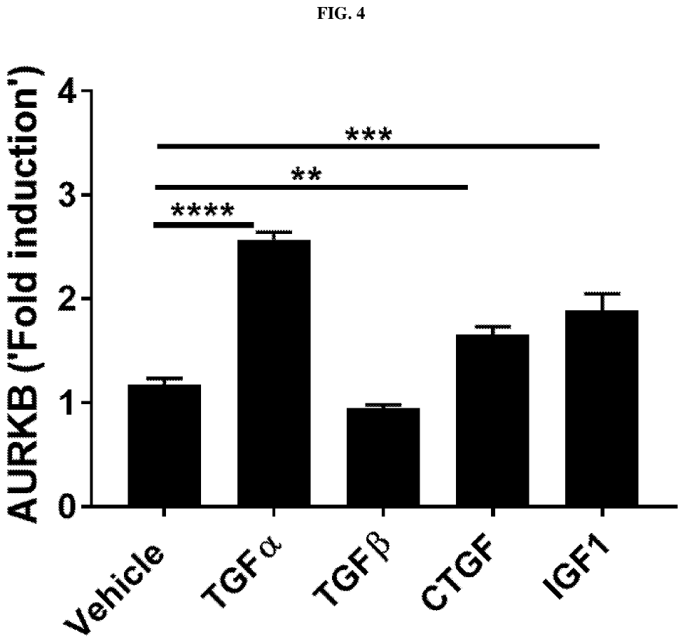

In some embodiments, the AURKB inhibitor can be AD6 (4-[(5-bromo-1,3-thiazol-2-yl)amino]-N-methyl-benzamide); AJI-100 (N4-(2-Chlorophenyl)-N2-(4-carbamoyl)-5-fluoropyrimidine-2,4-diamine; see FIG. 4 in YANG et al. (2014) “Dual Aurora A and JAK2 kinase blockade effectively suppresses malignant transformation” Oncotarget, Vol. 5, No. 10, pp. 2947-2961, which is herein incorporated by reference in its entirety); AJI-214 (N4-(phenyl)-N2-(4-carbamoyl)-5-fluoropyrimidine-2,4-diamine; see FIG. 4 in YANG et al. (2014) “Dual Aurora A and JAK2 kinase blockade effectively suppresses malignant transformation” Oncotarget, Vol. 5, No. 10, pp. 2947-2961, which is herein incorporated by reference in its entirety); AMG-900 (CAS number 945595-80-2; N-(4-(3-(2-aminopyrimidin-4-yl)pyridin-2-yloxy)phenyl)-4-(4-methylthiophen-2-yl)phthalazin-1-amine); AT9283 (CAS number 896466-04-9; 1-cyclopropyl-3-[(3Z)-3-[5-(morpholin-4-ylmethyl)benzimidazol-2-ylidene]-1,2-dihydropyrazol-4-yl]urea); HOWARD et al. (2009) “Fragment-based discovery of the pyrazol-4-yl urea (AT9283), a multitargeted kinase inhibitor with potent aurora kinase activity” J Med Chem, Vol. 52, No. 2, pp. 379-388, which is herein incorporated by reference in its entirety); Aurora Kinase Inhibitor II (AI II) (CAS number 331770-21-9; N-[4-[(6,7-dimethoxy-4-quinazolinyl)amino]phenyl]-benzamide); AZD1152 (CAS number 722543-31-9; 2-[ethyl-[3-[4-[[5-[2-(3-fluoroanilino)-2-oxoethyl]-1H-pyrazol-3-yl]amino]quinazolin-7-yl]oxypropyl]amino]ethyl dihydrogen phosphate); Barasertib (also known as AZD1152-HQPA or AZD2811) (CAS number 722544-51-6; 3-[[7-[3-[Ethyl(2-hydroxyethyl)amino]propoxy]-4-quinazolinyl]amino]-N-(3-fluorophenyl)-1H-pyrazole-5-acetamide); BI-811283 (see Sini et al., (2016) “Pharmacological Profile of BI 847325, an Orally Bioavailable, ATP-Competitive Inhibitor of MEK and Aurora Kinases” Mol Cancer Ther; Vol. 15, No. 10, pp. 2388-2398 (Supplementary Figure S1) which is herein incorporated by reference in its entirety; 4-((4-(((1R,25)-2-(isopropylcarbamoyl)cyclopentyl)amino)-5-(trifluoromethyl)pyrimidin-2-yl)amino)-N-methyl-N-(1-methylpiperidin-4-yl)benzamide); BMS-754807 (CAS number 1001350-96-4; (2S)-1-[4-[(5-Cyclopropyl-1H-pyrazol-3-yl)amino]pyrrolo[2,1-f][1,2,4]triazin-2-yl]-N-(6-fluoro-3-pyridinyl)-2-methyl-2-Pyrrolidinecarboxamide); CCT129202 (CAS number 942947-93-5; 2-(4-(6-chloro-2-(4-(dimethylamino)phenyl)-3H-imidazo[4,5-b]pyridin-7-yl)piperazin-1-yl)-N-(thiazol-2-yl)acetamide); Chiauranib (CAS number 1256349-48-0; N-(2-aminophenyl)-6-((7-methoxyquinolin-4-yl)oxy)-1-naphthamide); CYC116 (CAS number 693228-63-6; 4-methyl-5-(2-(4-morpholinophenylamino)pyrimidin-4-yl)thiazol-2-amine); ENMD-2076 (CAS number 934353-76-1; (E)-N-(5-methyl-1H-pyrazol-3-yl)-6-(4-methylpiperazin-1-yl)-2-styrylpyrimidin-4-amine); GSK-1070916 (CAS number 942918-07-2; 3-(4-(4-(2-(3-((dimethylamino)methyl)phenyl)-1H-pyrrolo[2,3-b]pyridin-4-yl)-1-ethyl-1H-pyrazol-3-yl)phenyl)-1,1-dimethylurea); Hesperadin (CAS number 422513-13-1; N-[(3Z)-2-Oxo-3-[phenyl-[4-(piperidin-1-ylmethyl)anilino]methylidene]-1H-indol-5-yl]ethanesulfonamide); Ilorasertib (aka ABT-348; CAS number 1227939-82-3; 1-(4-(4-amino-7-(1-(2-hydroxyethyl)-1H-pyrazol-4-yl)thieno[3,2-c]pyridin-3-yl)phenyl)-3-(3-fluorophenyl)urea); JNJ-7706621 (CAS number 443797-96-4; 4-[5-amino-1-(2,6-difluoro-benzoyl)-1H-[1,2,4]triazol-3-ylamino]-benzenesulfonamide); KW-2449 (CAS number 1000669-72-6; (E)-(4-(2-(1H-indazol-3-yl)vinyl)phenyl)(piperazin-1-yl)methanone); KW-2450 (CAS number 904899-25-8; (E)-N-(2-(2-(1H-indazol-3-yl)vinyl)-5-((4-(2-hydroxyacetyl)piperazin-1-yl)methyl)phenyl)-3-methylthiophene-2-carboxamide 4-methylbenzenesulfonate) or its tosylate salt; MK-6592 (aka VX667; see BOSS et al. (2009) “Clinical Experience with Aurora Kinase Inhibitors: A Review” The Oncologist Vol. 14, pp. 780-793, which is herein incorporated by reference in its entirety; (S)-(5-chloro-2-fluorophenyl)(3-(4-(3-cyclopropyl-3-fluoroazetidin-1-yl)-6-(3-methyl-1H-pyrazol-5-ylamino)pyrimidin-2-yloxy)pyrrolidin-1-yl)methanone); MLN8054 (CAS number 869363-13-3; 4-((9-chloro-7-(2,6-difluorophenyl)-5H-benzo[c]pyrimido[4,5-e]azepin-2-yl)amino)benzoic acid); MLN8237 (CAS number 1028486-01-2; 4-{[9-Chloro-7-(2-fluoro-6-methoxyphenyl)-5H-pyrimido[5,4-d][2]benzazepin-2-yl]amino}-2-methoxybenzoic acid); PF-03814735 (CAS number 942487-16-3; N-[2-[(1S,4R)-6-[[4-(Cyclobutylamino)-5-(trifluoromethyl)-2-pyrimidinyl]amino]-1,2,3,4-tetrahydronaphthalen-1,4-imin-9-yl]-2-oxoethyl]acetamide) or its mesylate salt; PHA-680632 (CAS number 398493-79-3; N-(2,6-diethylphenyl)-3-[[4-(4-methylpiperazin-1-yl)benzoyl]amino]-4,6-dihydro-1H-pyrrolo [3,4-c]pyrazole-5-carboxamide); PHA-739358 (aka Danusertib; CAS number 827318-97-8; 4-(4-methyl-1-piperazinyl)-N-[1,4,5,6-tetrahydro-5-[(2R)-2-methoxy-2-phenylacetyl]pyrrolo[3,4-c]pyrazol-3-yl]-benzamide); SNS314 (CAS number 1146618-41-8; 1-(3-chlorophenyl)-3-[5-[2-(thieno[3,2-d]pyrimidin-4-ylamino)ethyl]-1,3-thiazol-2-yl]urea) or its mesylate salt; SU6668 (CAS number 252916-29-3; 5-[1,2-Dihydro-2-oxo-3H-indol-3-ylidene)methyl]-2,4-dimethyl-1H-pyrrole-3-propanoic acid); TAK-901 (CAS number 934541-31-8; 5-(3-(ethylsulfonyl)phenyl)-3,8-dimethyl-N-(1-methylpiperidin-4-yl)-9H-pyrido[2,3-b]indole-7-carboxamide); TX47 (3,3′-((1H-indole-2,3-diyl)bis(methylene))bis(1H-indole); and the other inhibitors disclosed in WO2018086584 A1, which is herein incorporated by reference in its entirety); TY-011 (9-(2-chloro-phenyl)-6-ethyl-1-methyl-2,4-dihydro-2,3,4,7,10-pentaaza-benzo[f]azulene; see FIG. 1 in LIU et al. (2016) “Antitumor activity of TY-011 against gastric cancer by inhibiting Aurora A, Aurora B and VEGFR2 kinases” J Exp Clin Cancer Res., Vol. 35, Article 183, which is herein incorporated by reference in its entirety); VX-680 (aka Tozasertib; CAS number 639089-54-6; N-[4-[4-(4-Methylpiperazin-1-yl)-6-[(5-methyl-1H-pyrazol-3-yl)amino]pyrimidin-2-yl]sulfanylphenyl]cyclopropanecarboxamide); ZM447439 (CAS number 331771-20-1; N-[4-[[6-methoxy-7-[3-(4-morpholinyl)propoxy]-4-quinazolinyl]amino]phenyl]-benzamide); or a salt, ester, or solvate of any of the aforementioned.

In some embodiments, the AURKB inhibitor can be AD6; AJI-100; AJI-214; AT9283; Aurora Kinase Inhibitor II (AI II); AZD1152; Barasertib (aka AZD1152-HQPA); BMS-754807; CCT129202; CYC116; hesperadin; JNJ-7706621; KW-2450 or its tosylate salt; MLN8054; MLN8237; PF-03814735 or its mesylate salt; PHA-680632; PHA-739358; SNS314 or its mesylate salt; SU6668; TX47; TY-011; VX-680; or ZM447439; or a salt, ester, or solvate of any of the aforementioned.

In some embodiments, the AURKB inhibitor can be AD6; AJI-100; AJI-214; AT9283; Aurora Kinase Inhibitor II (AI II); AZD1152; Barasertib (aka AZD1152-HQPA); BMS-754807; CCT129202; CYC116; hesperadin; JNJ-7706621; KW-2450 or its tosylate salt; MLN8054; MLN8237; PF-03814735 or its mesylate salt; PHA-680632; PHA-739358; SNS314 or its mesylate salt; SU6668; TX47; TY-011; VX-680; or ZM447439.

In other embodiments, the AURKB inhibitor can be AMG-900; AT-9283; AZD1152; Barasertib; BI-811283; Chiauranib; CYC-116; ENMD-2076; GSK-1070916; Ilorasertib; KW-2449; MK-6592; PF-03814735 or its mesylate salt; PHA-739358 (aka Danusertib); TAK-901; SNS-314 or its mesylate salt; or VX-680 (aka Tozasertib); or a salt, ester, or solvate of any of the aforementioned.

In other embodiments, the AURKB inhibitor can be AMG-900; AT-9283; AZD1152; Barasertib; BI-811283; Chiauranib; CYC-116; ENMD-2076; GSK-1070916; Ilorasertib; KW-2449; MK-6592; PF-03814735 or its mesylate salt; PHA-739358 (aka Danusertib); TAK-901; SNS-314 or its mesylate salt; or VX-680 (aka Tozasertib).

In still other embodiments, the AURKB inhibitor can be AD6; AJI-100; AJI-214; AT9283; Aurora Kinase Inhibitor II (AI II); AZD1152; Barasertib (aka AZD1152-HQPA); BMS-754807; CYC116; hesperadin; JNJ-7706621; KW-2450 or its tosylate salt; PF-03814735 or its mesylate salt; TX47; TY-011; or ZM447439; or a salt, ester, or solvate of any of the aforementioned.

In some embodiments, the AURKB inhibitor can be AD6; AJI-100; AJI-214; AT9283; Aurora Kinase Inhibitor II (AI II); AZD1152; Barasertib (aka AZD1152-HQPA); BMS-754807; CYC116; hesperadin; JNJ-7706621; KW-2450 or its tosylate salt; PF-03814735 or its mesylate salt; TX47; TY-011; or ZM447439.

In other embodiments, the AURKB inhibitor can be barasertib (AZD1152-HQPA) or AZD1152, or a salt, ester, or solvate of barasertib or of AZD1152.

In yet other embodiments, the AURKB inhibitor can be barasertib (AZD1152-HQPA) or AZD1152.

In some embodiments, the AURKB inhibitor can be in the form of a salt, an ester, or a solvate. In other embodiments, the AURKB inhibitor can be in various forms, such as uncharged molecules, components of molecular complexes, or non-irritating pharmacologically acceptable salts, including but not limited to hydrochloride, hydrobromide, sulphate, phosphate, dihydrogen phosphate, nitrate, borate, acetate, maleate, tartrate, salicylate, tosylate, and mesylate. In some instances, for acidic compounds, salts can include metals, amines, or organic cations (e.g. quaternary ammonium). Esters can include any suitable esters such as but not limited to when an —OH group is replaced by an —O-alkyl group, where alkyl can be but is not limited to methyl, ethyl, propyl, or butyl. Solvates can include any suitable solvent (e.g., water, alcohols, ethanol) complexed (e.g., reversibly associated) with the molecule (e.g., AURKB inhibitor).

### Compositions Used for Treating

In certain embodiments, one or more AURKB inhibitors (e.g., barasertib or AZD1152) can be part of a composition and can be in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, or no more than about 99.99%, from about 0.0001% to about 99%, from about 0.0001% to about 50%, from about 0.01% to about 95%, from about 1% to about 95%, from about 10% to about 90%, or from about 25% to about 75%.

In some embodiments, one or more AURKB inhibitors (e.g., barasertib or AZD1152) can be purified or isolated in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.0001% to about 99%, from about 0.0001% to about 50%, from about 0.01% to about 95%, from about 1% to about 95%, from about 10% to about 90%, or from about 25% to about 75%.

Some embodiments of the present invention include compositions comprising one or more AURKB inhibitors (e.g., barasertib or AZD1152). In certain embodiments, the composition is a pharmaceutical composition, such as compositions that are suitable for administration to animals (e.g., mammals, primates, monkeys, humans, canine, feline, porcine, mice, rabbits, or rats). In some instances, the pharmaceutical composition is non-toxic, does not cause side effects, or both. In some embodiments, there may be inherent side effects (e.g., it may harm the patient or may be toxic or harmful to some degree in some patients).

An effective amount (e.g., a therapeutically effective amount) can be administered in one or more administrations. For some purposes of this invention, a therapeutically effective amount is an amount appropriate to treat an indication. By treating an indication is meant achieving any desirable effect, such as one or more of palliate, ameliorate, stabilize, reverse, slow, or delay disease progression, increase the quality of life, or to prolong life. Such achievement can be measured by any suitable method, such as measurement of the amount of fibrosis, the number of fibrocytes, the number of fibroblasts, the number of myofibroblasts, the extent of subpleural lung thickening, lung weight, body weight, lung function, or any suitable method to assess the progression of pulmonary fibrosis.

In some embodiments, one or more AURKB inhibitors (e.g., barasertib or AZD1152) can be part of a pharmaceutical composition and can be in an amount of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.001% to about 99%, from about 0.001% to about 50%, from about 0.1% to about 99%, from about 1% to about 95%, from about 10% to about 90%, or from about 25% to about 75%. In some embodiments, the pharmaceutical composition can be presented in a dosage form which is suitable for the topical, subcutaneous, intrathecal, intraperitoneal, oral, parenteral, rectal, cutaneous, nasal, vaginal, or ocular administration route. In other embodiments, the pharmaceutical composition can be presented in a dosage form which is suitable for parenteral administration, a mucosal administration, intravenous administration, depot injection (e.g., solid or oil based), subcutaneous administration, topical administration, intradermal administration, oral administration, sublingual administration, intratracheal administration, intranasal administration, or intramuscular administration. In some embodiments, the pharmaceutical composition can be presented in a dosage form which is suitable for an intratracheal administration, an intranasal administration, an aerosol administration, a nebulizer administration, a pressurized metered-dose inhaler (pMDI) administration, an inhaler administration, or a dry powder inhaler (DPI) administration. The pharmaceutical composition can be in any suitable form, for example but not limited to, tablets, capsules, pills, powders, granulates, suspensions, emulsions, solutions, gels (including hydrogels), pastes, ointments, creams, plasters, drenches, delivery devices, suppositories, enemas, injectables, implants, sprays, aerosols or other suitable forms.

In some embodiments, the pharmaceutical composition can include one or more formulary ingredients. A “formulary ingredient” can be any suitable ingredient (e.g., suitable for the drug(s), for the dosage of the drug(s), for the timing of release of the drugs(s), for the disease, for the disease state, or for the delivery route) including, but not limited to, water (e.g., boiled water, distilled water, filtered water, pyrogen-free water, or water with chloroform), sugar (e.g., sucrose, glucose, mannitol, sorbitol, xylitol, or syrups made therefrom), ethanol, glycerol, glycols (e.g., propylene glycol), acetone, ethers, DMSO, surfactants (e.g., anionic surfactants, cationic surfactants, zwitterionic surfactants, or nonionic surfactants (e.g., polysorbates)), oils (e.g., animal oils, plant oils (e.g., coconut oil or arachis oil), or mineral oils), oil derivatives (e.g., ethyl oleate, glyceryl monostearate, or hydrogenated glycerides), excipients, preservatives (e.g., cysteine, methionine, antioxidants (e.g., vitamins (e.g., A, E, or C), selenium, retinyl palmitate, sodium citrate, citric acid, chloroform, or parabens, (e.g., methyl paraben or propyl paraben)), or combinations thereof. For example, a depot injection (e.g., solid or oil based) could include one or more formulary ingredients.

In certain embodiments, pharmaceutical compositions can be formulated to release the one or more AURKB inhibitors (e.g., barasertib or AZD1152) substantially immediately upon the administration or any substantially predetermined time or time after administration. Such formulations can include, for example, controlled release formulations such as various controlled release compositions and coatings. For example, a depot injection (e.g., solid or oil based) could be used for a controlled release (e.g., of barasertib or of AZD1152), and in some instances, could be injected once per month (or once per day, once per week, once per three months, once per six months, or once per year).

Other formulations (e.g., formulations of a pharmaceutical composition) can, in certain embodiments, include those incorporating the drug (or control release formulation) into food, food stuffs, feed, or drink. For example, barasertib or AZD1152 could be administered orally once per day, twice per day, three times per day, once per two days, or once per week.

Some embodiments of the invention can include methods of treating an organism for fibrosis. In certain embodiments, treating comprises administering at least one AURKB inhibitor. In other embodiments, treating comprises administering at least one AURKB inhibitor to an animal that is effective to treat fibrosis. In some embodiments, a composition or pharmaceutical composition comprises at least one AURKB inhibitor which can be administered to an animal (e.g., mammals, primates, monkeys, or humans) in an amount of about 0.005 to about 100 mg/kg body weight, about 0.005 to about 50 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 0.5 mg/kg, about 1 mg/kg, about 3 mg/kg, about 5 mg/kg, about 5.5 mg/kg, about 6 mg/kg, about 6.5 mg/kg, about 7 mg/kg, about 7.5 mg/kg, about 8 mg/kg, about 10 mg/kg, about 12 mg/kg, or about 15 mg/kg. In regard to some conditions, the dosage can be about 0.5 mg/kg human body weight, about 5 mg/kg human body weight, about 6.5 mg/kg human body weight, about 10 mg/kg human body weight, about 50 mg/kg human body weight, about 80 mg/kg human body weight, or about 100 mg/kg human body weight. In some instances, some animals (e.g., mammals, mice, rabbits, feline, porcine, or canine) can be administered a dosage of about 0.005 to about 100 mg/kg body weight, about 0.005 to about 50 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 1 mg/kg, about 5 mg/kg, about 10 mg/kg, about 20 mg/kg, about 30 mg/kg, about 40 mg/kg, about 50 mg/kg, about 80 mg/kg, about 100 mg/kg, or about 150 mg/kg. Of course, those skilled in the art will appreciate that it is possible to employ many concentrations in the methods of the present invention, and using, in part, the guidance provided herein, will be able to adjust and test any number of concentrations in order to find one that achieves the desired result in a given circumstance. In other embodiments, the AURKB inhibitor can be administered in combination with one or more other therapeutic agents to treat a given fibrosis.

In some embodiments, the compositions can include a unit dose of one or more AURKB inhibitors in combination with a pharmaceutically acceptable carrier and, in addition, can include other medicinal agents, pharmaceutical agents, carriers, adjuvants, diluents, and excipients. In certain embodiments, the carrier, vehicle or excipient can facilitate administration, delivery and/or improve preservation of the composition. In other embodiments, the one or more carriers, include but are not limited to, lactose powder or saline solutions such as normal saline, Ringer's solution, PBS (phosphate-buffered saline), and generally mixtures of various salts including potassium and phosphate salts with or without sugar additives such as glucose. Carriers can include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents. In other embodiments, the one or more excipients can include, but are not limited to water, saline, dextrose, glycerol, ethanol, lactose powder or the like, and combinations thereof. Nontoxic auxiliary substances, such as wetting agents, buffers, or emulsifiers may also be added to the composition. Formulations (e.g., oral formulations) can include such normally employed excipients as, for example, pharmaceutical grades of mannitol, lactose, starch, magnesium stearate, sodium saccharine, cellulose, and magnesium carbonate.

The presently-disclosed subject matter is further illustrated by the following specific but non-limiting examples. The following examples may include compilations of data that are representative of data gathered at various times during the course of development and experimentation related to the present invention.

### EXAMPLES

### Materials and Methods

### Pathway Based Drug Discovery and Computational Analysis

Differential gene expression signatures of IPF lungs from human patients (6 independent cohorts; >300 IPF patients and ˜100 control) were queried against the LINCS database to obtain a ranked list of candidate therapeutics based on the strength of their “connectivity scores” (SUBRAMANIAN et al. (2017) “A Next Generation Connectivity Map: L1000 Platform and the First 1,000,000 Profiles” Cell, Vol. 171, pp. 1437-1452, article e1417, which is herein incorporated by reference in its entirety; LAMB (2007) “The Connectivity Map: a new tool for biomedical research” Nat Rev Cancer, Vol. 7, pp. 54-60, which is herein incorporated by reference in its entirety; LAMB et al. (2006) “The Connectivity Map: using gene-expression signatures to connect small molecules, genes, and disease” Science, Vol. 313, pp. 1929-1935, which is herein incorporated by reference in its entirety). The LINCS Cloud query API (<<http://apps.lincscloud.org/query>>) and Kolmogorov-Smirnov-test (KS test) based algorithm (LAMB et al. (2006) “The Connectivity Map: using gene-expression signatures to connect small molecules, genes, and disease” Science, Vol. 313, pp. 1929-1935) was used to rank candidate compounds. The LINCScloud API offers programmatic access to annotations and perturbational signatures in the LINCS L1000 dataset (Broad-Institute. Library of Integrated Cellular Signatures (LINCS). 2014. Available from: <<http://www.lincsproject.org/>>, which is herein incorporated by reference in its entirety) via a collection of HTTP-based RESTful web services. The Pattern-Matching Software searches for two-directional matches, considering both the up and down IPF gene sets, in comparing the query against signatures (z-scored differential expressions) in the LINCS L1000 dataset. The system will then generate a list of signatures rank ordered by the strength of the match to the query. To make the small molecule predictions more robust and informed, we also employed a systems biology-based approach. To do this, we combined prior knowledge of IPF-centered biological processes and pathways with IPF transcriptomic signatures to build pathway-specific subnetworks or functional modules. These IPF-pathway-centric gene signatures were then used to query LINCS to identify pathway-specific ranked list of compounds. In the final step, we applied meta-analysis to these IPF-specific individual ranked lists of compounds to identify (a) top compounds and (b) compounds that are consistently ranked among the best or are occurring in more than one list. Barasertib (AZD1152-HQPA), a known AURKB inhibitor, was among the top compounds along with other tyrosine kinase inhibitors (such as Nintedanib, approved drug for IPF) (SONTAKE et al. (2017) “Hsp90 regulation of fibroblast activation in pulmonary fibrosis” JCI Insight, Vol. 2, Issue 4, Article e91454. <<https://doi.org/10.1172/jci.insight.91454>>, which is herein incorporated by reference in its entirety). AZD1152 is an orally bioavailable, small-molecule, dihydrogen phosphate prodrug of the pyrazoloquinazoline aurora kinase inhibitor AZD1152-hydroxyquinazoline pyrazol anilide (AZD1152-HQPA) with potential antineoplastic activity. Upon administration of AZD1152 and rapid conversion from the prodrug form in plasma to AZD1152-HQPA (barasertib), AZD1152-HQPA specifically binds to and inhibits Aurora kinase B. For network representation of select significantly enriched biological processes by barasertib, Cytoscape application (SHANNON et al., (2003) “Cytoscape: a software environment for integrated models of biomolecular interaction networks” Genome Res, Vol. 13, pp. 2498-2504, which is herein incorporated by reference in its entirety) was used.

Barasertib (AZD1152-HQPA) was obtained from Selleckchem (Houston, Tex.).

Immunohistochemistry Lungs were inflated and fixed using 10% buffered formalin and embedded in paraffin. The lung sections were prepared and stained with H&E or Mason's trichrome as described previously (SONTAKE et al. (2017) “Hsp90 regulation of fibroblast activation in pulmonary fibrosis” JCI Insight, Vol. 2, Issue 4, Article e91454. <<https://doi.org/10.1172/jci.insight.91454>>, which is herein incorporated by reference in its entirety). For immunostainings, the lung sections were stained with antibodies against AURKB (mouse monoclonal anti-human AURKB, Abcam, Cambridge, Mass., USA), and αSMA (Clone 1A4, Dako, Calif., USA), as described previously (MADALA et al. (2014) “Bone marrow-derived stromal cells are invasive and hyperproliferative and alter transforming growth factor-alpha-induced pulmonary fibrosis” Am J Respir Cell Mol Biol, Vol. 50, pp. 777-786, which is herein incorporated by reference in its entirety).

Mouse model of TGFα-induced pulmonary fibrosis and Barasertib treatment therapy The generation of TGFα-overexpressing mice has been described previously (HARDIE et al. (2004) “Conditional expression of transforming growth factor-alpha in adult mouse lung causes pulmonary fibrosis” Am J Physiol Lung Cell Mol Physiol, Vol. 286, pp. L741-749, which is herein incorporated by reference in its entirety). Clara cell-specific protein-rtTA^{+/-} (CCSP-rtTA) mice were crossed with heterozygous (TetO)₇-cmv TGFα mice to produce bitransgenic CCSP/TGFα mice. To induce TGFα expression, the transgenic mice were fed with doxycycline (Dox)-containing chow (62.5 mg/kg) (MADALA et al. (2014) “Inhibition of the alphavbeta6 integrin leads to limited alteration of TGF-alpha-induced pulmonary fibrosis” Am J Physiol Lung Cell Mol Physiol, Vol. 306, pp. L726-735, which is herein incorporated by reference in its entirety). They were housed under specific pathogen-free conditions and handled in accordance with protocols approved by the Institutional Animal Care and Use Committee of the Cincinnati Children's Hospital Research Foundation. Barasertib (Selleckchem, Houston, Tex.) was prepared in fresh vehicle (5% DMSO and 50% PEG in PBS) every day before treatment. Fibrosis was induced by overexpressing TGFα for 4 weeks, and mice were treated simultaneously with vehicle or barasertib (40 mg/kg, twice a day) was administered by intraperitoneal injections as described (MADALA et al. (2016) “p70 ribosomal S6 kinase regulates subpleural fibrosis following transforming growth factor-alpha expression in the lung” Am J Physiol Lung Cell Mol Physiol, Vol. 310, pp. L175-186, which is herein incorporated by reference in its entirety). Non-TGFα expressing mice on Dox treated with vehicle was used as a control group to determine extent of fibrosis in vehicle and barasertib treated groups.

Human and mouse lung primary mesenchymal cell cultures Human and mouse lung mesenchymal cell cultures were prepared as described (SONTAKE et al. (2017) “Hsp90 regulation of fibroblast activation in pulmonary fibrosis” JCI Insight, Vol. 2, Issue 4, Article e91454. <<https://doi.org/10.1172/jci.insight.91454>> (20 pages); SONTAKE et al. (2018) “Wilms' tumor 1 drives fibroproliferation and myofibroblast transformation in severe fibrotic lung disease” JCI Insight, Vol. 3, No. 16, Article e121252 <<https://doi.org/10.1172/jci.insight.121252>> (10 pages), which is herein incorporated by reference in its entirety). To isolate lung-resident fibroblasts, lung mesenchymal cells were harvested and incubated with anti-CD45 microbeads on ice for 15 min (Miltenyi Biotec, Auburn, Calif.). After washing twice with sterile buffer, cells were loaded onto magnetic columns (Miltenyi Biotec) and eluted with appropriate amounts of sterile buffer in the presence and absence of a magnetic field to separate unbound cells (CD45^{-ve} cells; lung-resident (myo)fibroblasts) or those bound to the column (CD45^{+ve} cells; fibrocytes). Purity of mesenchymal cell subsets was determined using flow cytometry (≥96%) (MADALA et al. (2014) “Bone marrow-derived stromal cells are invasive and hyperproliferative and alter transforming growth factor-alpha-induced pulmonary fibrosis” Am J Respir Cell Mol Biol, Vol. 50, pp. 777-786, which is herein incorporated by reference in its entirety).

RNA extraction and real-time PCR Total RNA was prepared from isolated cells and lung tissue using RNeasy Mini Kit (Qiagen Sciences, Valencia, Calif.) as described (MADALA et al. (2012) “Resistin-like molecule alpha1 (Fizz1) recruits lung dendritic cells without causing pulmonary fibrosis” Respiratory research, Vol. 13, Article 51, which is herein incorporated by reference in its entirety). Complementary DNA was prepared, and real-time PCR performed using the CFX384 Touch Real-Time PCR detection system and SYBR green super mix (Bio-Rad, Hercules, Calif.). Target gene transcripts in each sample were normalized to mouse hypoxanthine guanine phosphoribosyl transferase (Hprt) or human beta-actin.

WT1 siRNA transfection studies Primary human or mouse fibroblast cells were transfected with stealth AURKB small interfering RNA (siRNA) or mouse AURKB siRNA or stealth control siRNA (Invitrogen) using the Lipofectamine 3000 Transfection kit (Invitrogen) according to the manufacturer's instructions. Primary lung-resident mesenchymal cells were separated from fibrocytes using anti-CD45 magnetic beads as described previously (MADALA et al. (2014) “Bone marrow-derived stromal cells are invasive and hyperproliferative and alter transforming growth factor-alpha-induced pulmonary fibrosis” Am J Respir Cell Mol Biol, Vol. 50, pp. 777-786, which is herein incorporated by reference in its entirety) and grown on 12-well plates to 90% confluence. Cells were transfected with siRNA using OptiMEM media containing no antibiotics. Transfected cells were harvested 72 h post transfection and used for RNA isolation and gene-expression analysis.

Western blot Total lung tissue or primary lung-resident fibroblasts treated with DMSO or barasertib were lysed using RIPA lysis buffer supplemented with protease and phosphatase inhibitors. Total protein was quantified using a BCA kit (Thermo Fisher Scientific, Waltham, Mass.), and an equal amount of protein from a soluble fraction was subjected to SDS-PAGE on a 4-12% gel as described (SINGH et al. (2017) “Repetitive intradermal bleomycin injections evoke T-helper cell 2 cytokine-driven pulmonary fibrosis” Am J Physiol Lung Cell Mol Physiol, Vol. 313, pp. L796-L806, which is herein incorporated by reference in its entirety). Quantification was performed using the volume integration function of the phosphor imager software, Multigage (Fujifilm, Valhalla, N.Y.) as described (MADALA et al. (2016) “Unique and Redundant Functions of p70 Ribosomal S6 Kinase Isoforms Regulate Mesenchymal Cell Proliferation and Migration in Pulmonary Fibrosis” Am J Respir Cell Mol Biol, Vol. 55, pp. 792-803, which is herein incorporated by reference in its entirety).

Incucyte ZOOM caspase 3/7 apoptotic assay Kinetic estimation of caspase 3/7 activity was performed using the real-time imaging system IncuCyte ZOOM (Essen BioScience, Ann Arbor, Mich.). Activation of caspase-3/7 in cells undergoing apoptotic death cleaved the caspase-3/7 substrate to produce nuclear green-fluorescence (Caspase-3/7 Green Apoptosis Assay Reagent [Essen Bioscience]). Primary lung-resident (myo)fibroblasts were prepared from normal or fibrotic lung tissue and cultured in a 12-well plate to 50-60% confluency. After growing overnight in low serum-containing MEM media, they had adapted to low-serum conditions. They were then treated with media containing either Caspase 3/7 Green Apoptosis Assay Reagent at a final concentration of 5 μM/mL or Caspase 3/7 Green Apoptosis Assay Reagent and anti-Fas antibody (BD Biosciences) at a final concentration of 250 ng/mL. Time-lapse fluorescence imaging was performed using the IncuCyte ZOOM system (Essen BioScience); 9 images per well at 20× magnification were collected every 2 h for 24-48 h. The average number of green objects produced by the apoptotic cells were measured using Incucyte ZOOM software 2015A.

BrdU proliferation assays Primary lung-resident fibroblast proliferation was assessed using a BrdU Cell Proliferation Assay Kit (Cell Signaling Technology, Denver, Colo.) as described (SONTAKE et al. (2017) “Hsp90 regulation of fibroblast activation in pulmonary fibrosis” JCI Insight, Vol. 2, Issue 4, Article e91454. <<https://doi.org/10.1172/jci.insight.91454>>, which is herein incorporated by reference in its entirety; SONTAKE et al. (2018) “Wilms' tumor 1 drives fibroproliferation and myofibroblast transformation in severe fibrotic lung disease” JCI Insight, Vol. 3, No. 16, Article e121252 <<https://doi.org/10.1172/jci.insight.121252>> (10 pages), which is herein incorporated by reference in its entirety). Briefly, primary lung-resident fibroblasts were treated with DMSO or barasertib (0.1, 1, 2, and 5 μM) for 24 h then incubated with BrdU labeling solution for another 24 h along with barasertib or DMSO. The cells were fixed 24 h after BrdU labeling, and immunodetection of BrdU was performed according to the manufacturer's protocol. Change in proliferation was calculated as fold difference over control by measuring absorbance at 450 nm.

Statistical analysis All data were analyzed using Prism (version 7.02; GraphPad, La Jolla, Calif.). Student's t-test was used to compare the two experimental groups. One-way ANOVA with Sidak's multiple comparison was used to compare the various experimental groups, and two-way ANOVA to compare the independent variables between groups. Data were considered statistically significant for p values less than 0.05.

### Results and Discussion

Identification of barasertib as a candidate therapeutic in IPF. Using integrative systems biology-based approaches and computational screening, we identified barasertib as a candidate therapeutic for IPF. Briefly, differential gene expression signatures of IPF lungs from human patients (6 independent cohorts; >300 IPF patients and ˜100 control) were queried against the LINCS database to obtain a ranked list of candidate therapeutics based on the strength of their “connectivity scores”. Barasertib, a known AURKB inhibitor, was among the top compounds along with other tyrosine kinase inhibitors (such as Nintedanib, approved drug for IPF). Currently, barasertib is being investigated for anti-cancer therapy. To further elucidate the role of barasertib as a candidate therapeutic for IPF, we performed a direct comparison of pathways between barasertib and IPF. We undertook a functional enrichment analysis of the anti-correlated gene sets between IPF lungs and barasertib-treated cells from the LINCS database (i.e., genes up in IPF and down in barasertib treatment and vice versa). The ToppFun application of the ToppGene Suite (CHEN et al. (2009) “ToppGene Suite for gene list enrichment analysis and candidate gene prioritization” Nucleic Acids Res, Vol. 37, pp. W305-311) was utilized for the enrichment analysis. As shown in FIG. 1, cell proliferation, migration, apoptosis, and ECM production—hallmarks of fibrosis—were among the enriched biological processes putatively modulated by barasertib.

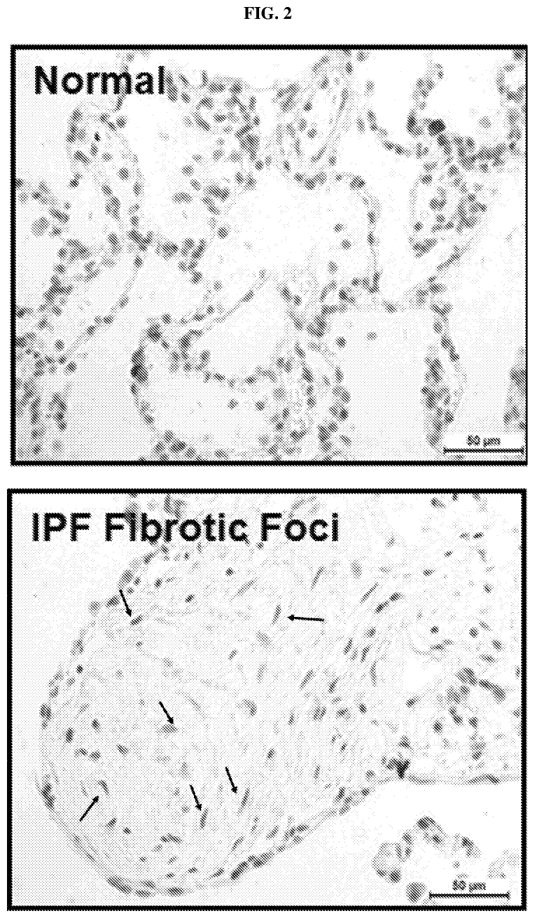

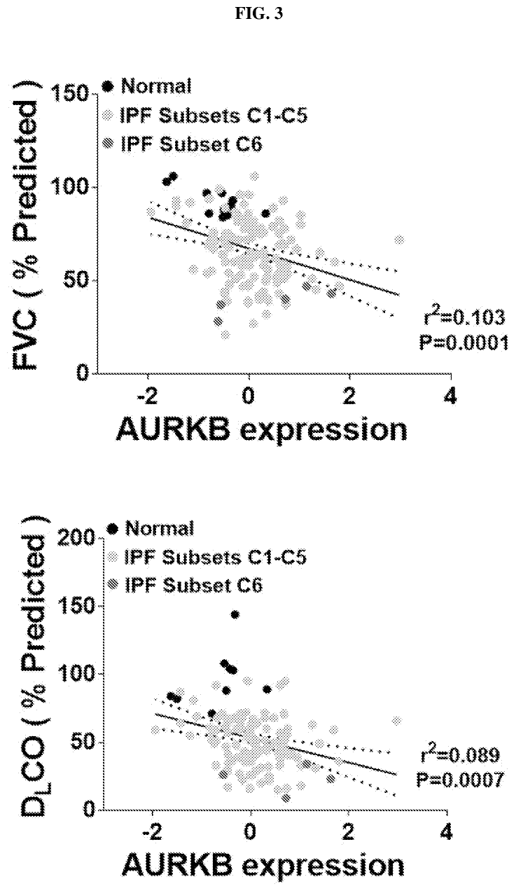

AURKB expression in human IPF. To determine therapeutic relevance of targeting aurora kinases, we immunostained IPF lung sections with antibodies and observed a marked increase in AURKB staining in spindle shaped fibroblasts located in subpleural regions and fibrotic foci of IPF lung tissue compared to normal lungs (FIG. 2). We previously characterized six different IPF subtypes using whole lung transcriptional profiles and lung function, combined with data-driven unsupervised clustering analysis to segregate normal from subtypes of IPF. To understand the possible pro-fibrotic roles of AURKB in IPF, we compared their expression levels with lung function parameters of mild to severe IPF and controls. We found that IPF subgroups showed heightened expression of AURKB and this increase is associated with decline in lung function (both FVC and D_{LCO}) (FIG. 3).

To determine direct effects of multiple pro-fibrotic growth factors in AURKB expression, we treated primary fibroblasts isolated from human lungs and treated with multiple growth factors including TGFα, TGFβ, CTGF, and IGF1. AURKB is upregulated in fibroblasts treated with TGFα, CTGF and IGF1 but not TGFβ (FIG. 4). Therefore, use of primary fibroblasts isolated from IPF lungs and a mouse model of TGFα-induced pulmonary fibrosis will allow us to further understand molecular activation of AURKB in fibrogenesis and also test inhibitors of AURKB that can mitigate fibroblast activation in pulmonary fibrosis.

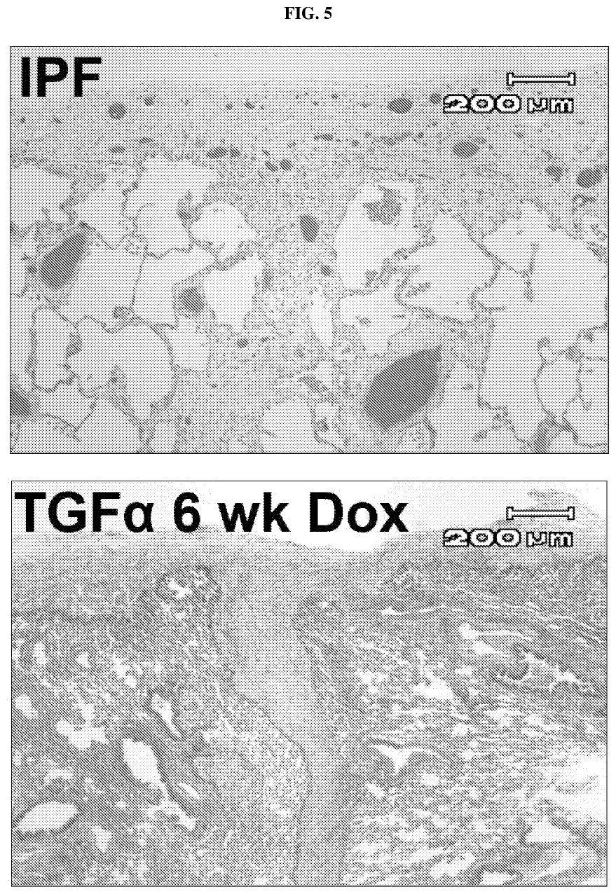

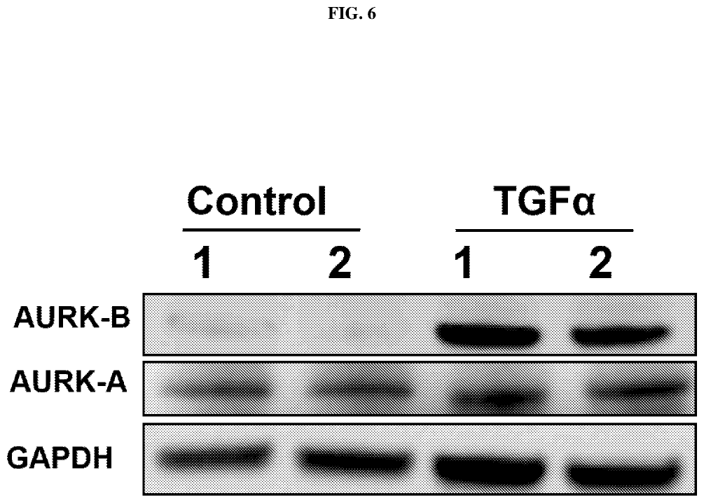

Mouse model of TGFα-induced fibrosis. EGFR (HER1) belongs to a receptor tyrosine-kinase protein family that also includes HER2/neu, HER3, and HER4. Six EGFR ligands including TGFα appear to have been identified in lungs or lung cells. EGFR and its ligands appear to be found in a number of cells in the lung including the alveolar and airway epithelium, fibroblasts, and macrophages. In the lung, EGFR appears to be activated both directly and indirectly by several inflammatory agents, including cytomegalovirus, endotoxin, tumor necrosis factor or TNF, and IL-13. Activation of EGFR appears to regulate diverse cellular functions, many of which are associated with fibrogenesis, and include cell growth, proliferation, differentiation, migration, and survival. Increases in the EGFR pathway appear to have been associated with a number of human fibrotic diseases. TGFα was reportedly detected in the lung lavage fluid of all 10 patients with IPF, but in none of 13 normal volunteers. It appears to have been demonstrated that an increase in TGFα and EGFR in IPF by immunohistochemistry with increased TGFα localized to type II epithelial cells, fibroblasts, and the vascular endothelium compared with controls. To further determine mechanisms of EGFR-mediated lung remodeling, transgenic mice were generated in which TGFα was conditionally overexpressed in the lung epithelium using the CCSP rtTA promoter, when mice are administered doxycycline (Dox). Overexpression of TGFα in the adult mouse causes progressive and extensive adventitial, interstitial, and subpleural fibrosis. Fibrosis occurred in the absence of inflammatory cell influx on lung histology or as measured by bronchoalveolar lavage cell counts and differential, or increased proinflammatory cytokines as measured from lung homogenates using ELISA or microarray analysis. Several histological features of fibrosis in the TGFα model can be found in the pathologic lesions of IPF, including subpleural fibrosis radiating into the adjoining interstitium and differentiation of myofibroblasts (FIG. 5). Physiologically, mice appear to develop progressive cachexia, changes in lung mechanics (increased airway resistance and elastance, as well as decreased lung compliance) and secondary pulmonary hypertension. Gene expression profiles after expression of TGFα were similar to IPF samples. AURKB is upregulated but not AURKA in the lungs of TGFα mice on Dox for 4 wks compared to fibroblasts from normal mouse lungs (FIG. 6). Therefore, the TGFα transgenic mouse is a model to further understand the role of AURKB in mediating pulmonary fibrogenesis and a tool to study therapeutics to reverse progressive pulmonary fibrosis.

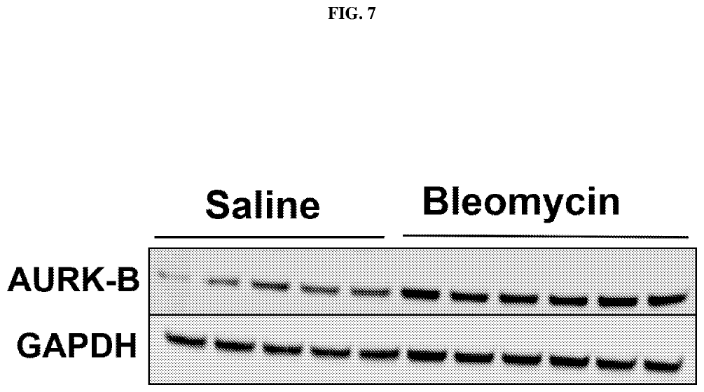

Mouse model of repetitive bleomycin-induced fibrosis. Bleomycin is a nonribosomal antibiotic peptide isolated from Streptomyces verticillatus. Bleomycin treatment induces DNA damage and reactive oxygen species generation. When lungs are exposed to bleomycin via the intratracheal route, mice appear to develop severe lung injury and the loss of the epithelial barrier that is marked by excessive tissue inflammation and fibrosis. Bleomycin-driven fibrotic responses appear to be short and reversible with limited or no significant changes in subpleural thickening and lung function. Therefore, we developed an alternative mouse model of bleomycin-induced pulmonary fibrosis. For these experiments, mice were injected intradermally with 100 μg of bleomycin for five days in a week for a total of 4 wks and these mice displayed a progressive decline in lung function with a greater than two-fold increase in airway resistance and lung hydroxyproline levels compared to saline-treated control mice. Repetitive intradermal administration of bleomycin resulted in mild inflammation, but extensive fibrosis that persisted for several weeks in subpleural and parenchymal areas of the lungs. Moreover, similarly to the TGFα model, we observed an increase in AURKB expression in the lungs of mice treated with bleomycin compared to saline (FIG. 7). Thus, we have established an alternative pre-clinical mouse model to confirm key findings of our anti-fibrotic therapies in reversing established pulmonary fibrosis.

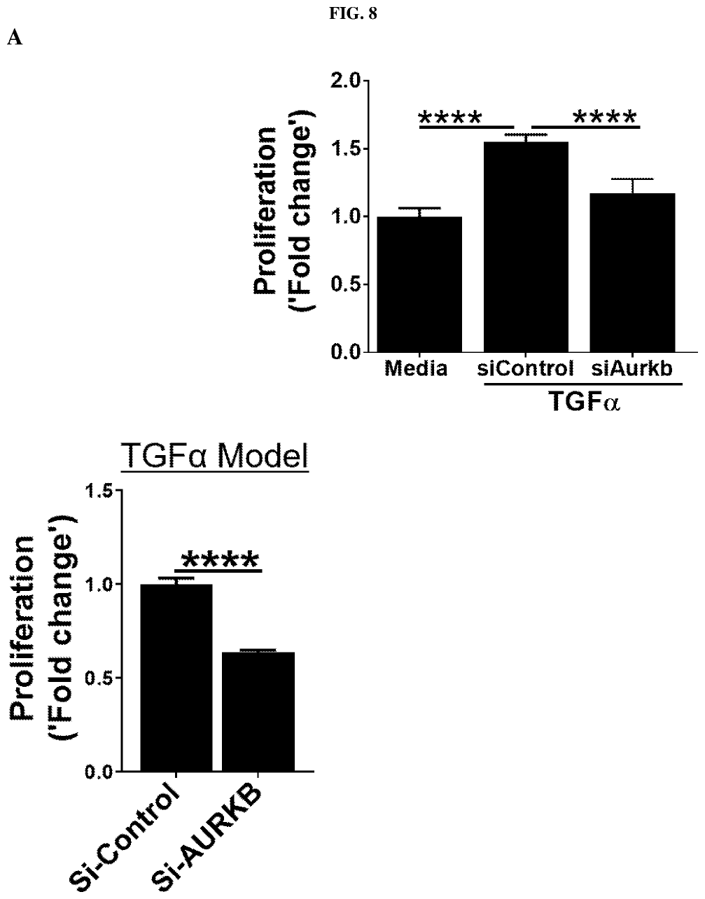

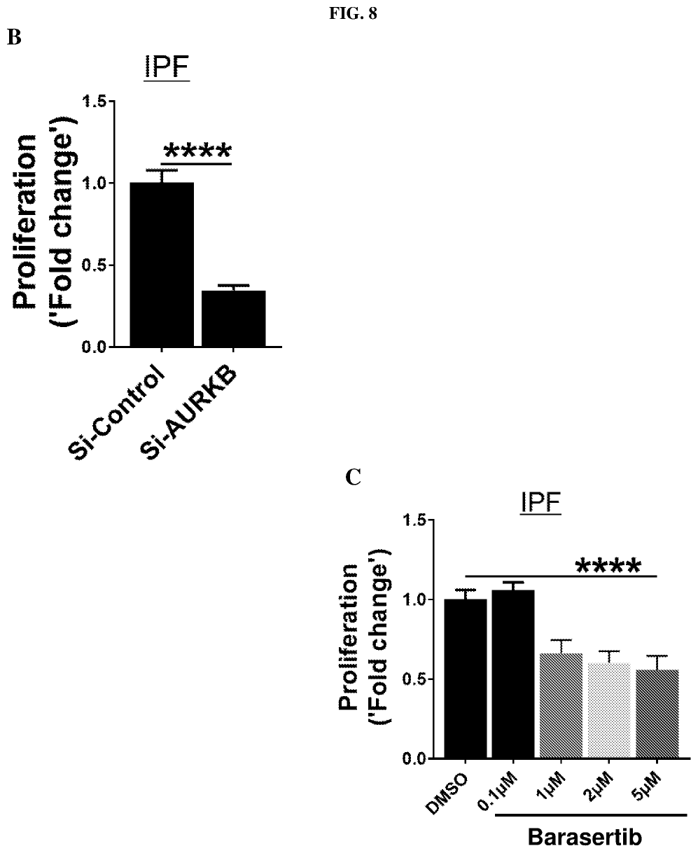

AURKB is a positive regulator of fibroproliferation. The proliferative expansion of lung-resident fibroblasts at the site of injury is a pathological process during initiation and progressive expansion of fibrotic lesions in the lung. To determine whether the loss of AURKB transcripts mitigate fibroproliferation, we treated lung-resident fibroblasts of TGFα mice or IPF with siRNA-specific to AURKB mRNA as well as controls siRNA for 72 hrs. Treatment of fibroblasts with AURKB siRNA were able to specifically knock down the corresponding AURKB expression compared to control siRNA (data not shown). Also, the loss of AURKB was sufficient to attenuate proliferation of lung-resident fibroblasts from TGFα model or IPF lungs compared to siRNA treated controls (FIG. 8A and FIG. 8B). Inhibition of AURKB phosphorylation resulted in a decrease in the proliferation of lung-resident fibroblasts isolated from IPF lungs in a dose-dependent manner, as the concentration of barasertib was increased from 0.1 μM to 5 μM (FIG. 8C).

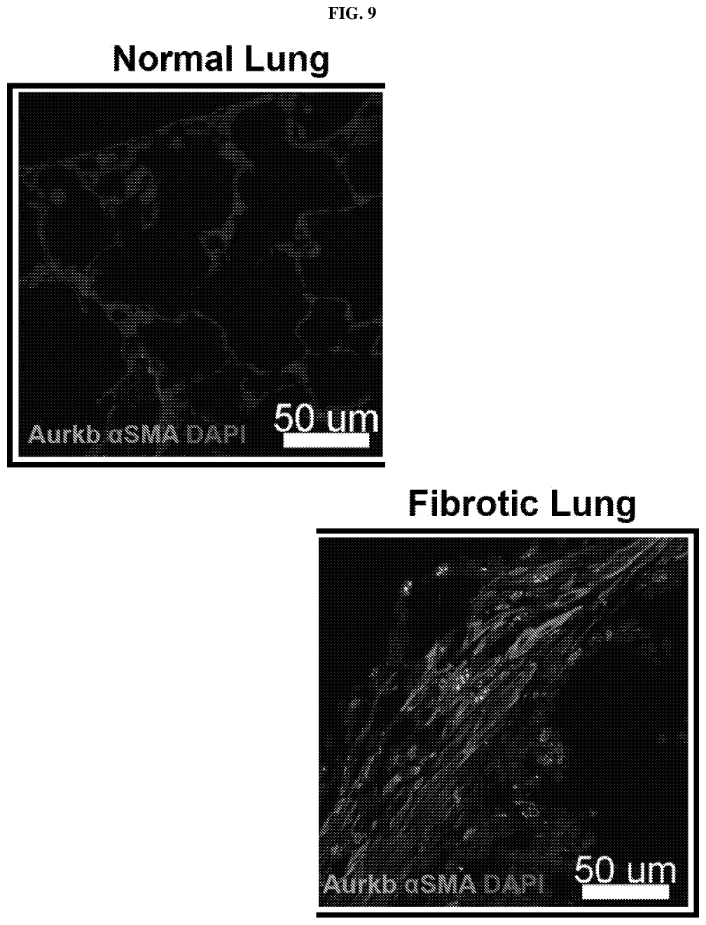

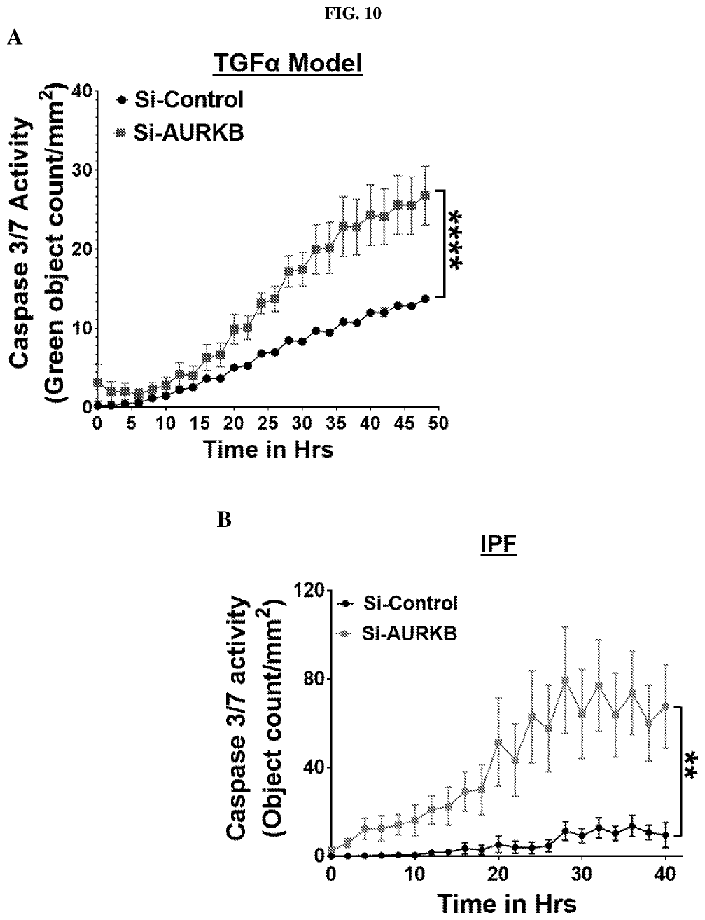

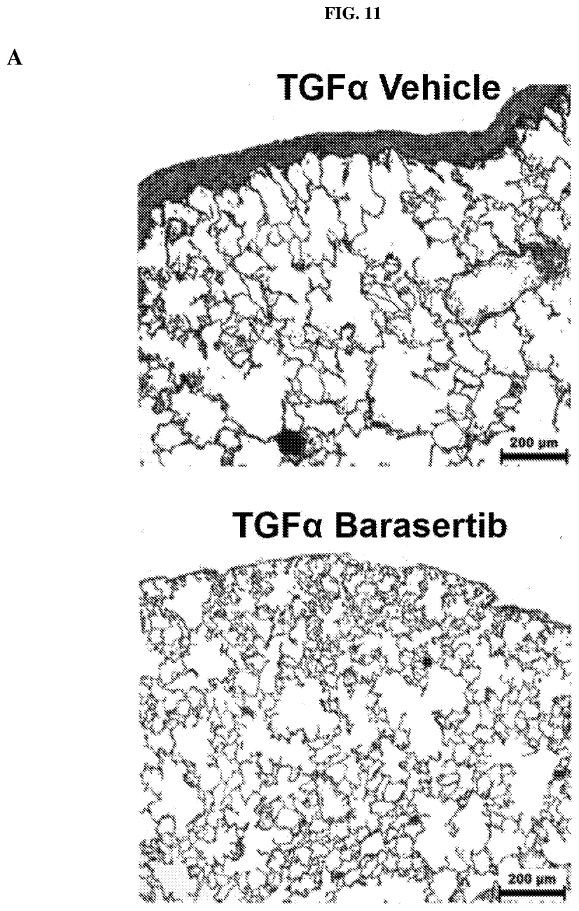

AURKB is a positive regulator of myofibroblast survival. The persistence of myofibroblasts in injured lung tissue is a cause for non-resolving fibrosis. In some instances, the successful resolution of fibrosis is not only dependent on inhibiting myofibroblast differentiation but eliminating apoptosis-resistant (myo)fibroblasts. To determine if AURKB is expressed in myofibroblasts, we co-immunostained lung sections of TGFα mice on Dox and observed an increase in AURKB-positive myofibroblasts that accumulate in the mature fibrotic lesions of TGFα mice compared to control mice on Dox for 4 wks (FIG. 9). Therefore, we postulated that inhibition of AURKB expression is sufficient to attenuate myofibroblast survival. To test this hypothesis, we treated lung-resident (myo)fibroblasts isolated from fibrotic lesions of TGFα mice on Dox for four wks or IPF lungs with siRNA-specific to AURKB mRNA as well as controls siRNA for 48 hrs. FasL-induced apoptosis was quantified using Essen BioScience IncuCyte™ FLR or ZOOM that acquired live images of cells undergoing caspase-3/7 mediated apoptosis. The loss of AURKB transcripts was sufficient to induce apoptosis in lung-resident (myo)fibroblasts isolated from fibrotic lesions of TGFα mice on Dox for four wks or IPF lungs (FIG. 10). Similarly, inhibition of AURKB phosphorylation with barasertib resulted in an increase in apoptotic clearance of lung-resident (myo)fibroblast (data not shown).

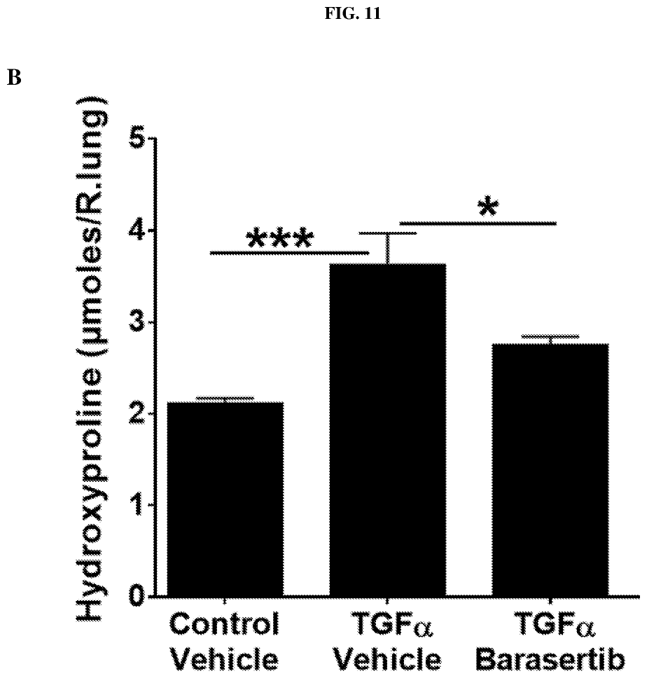

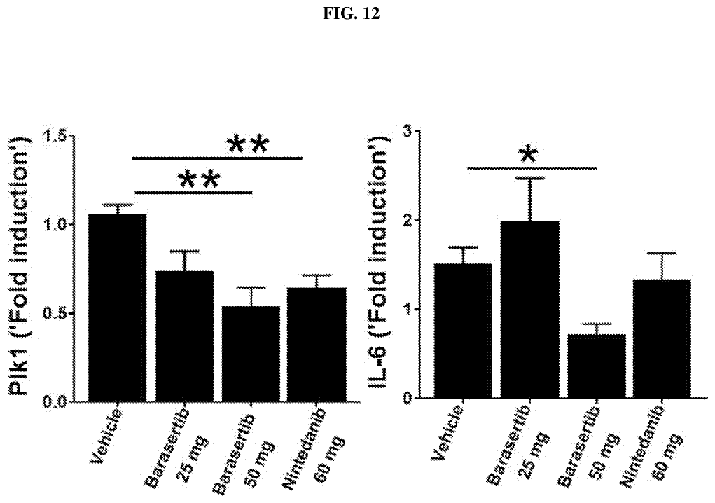

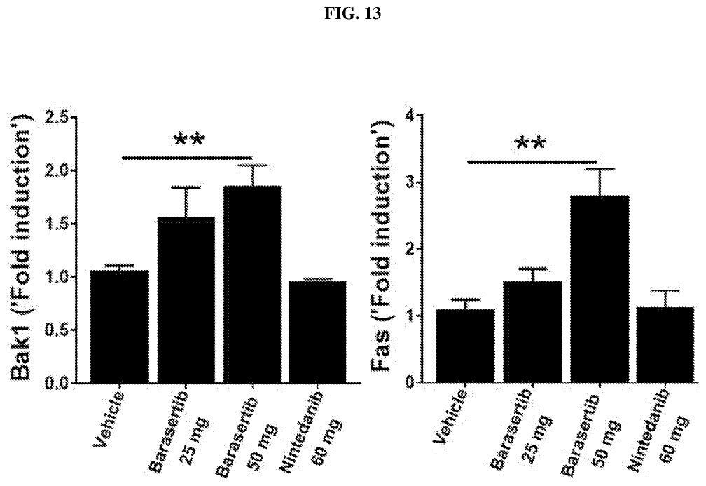

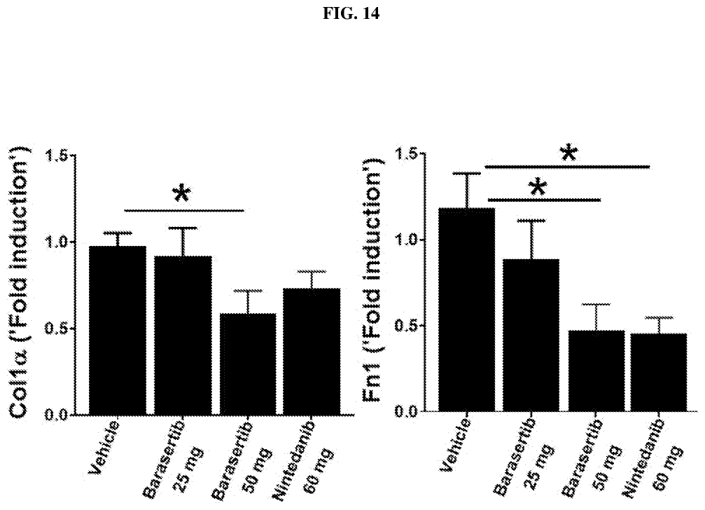

Pharmacological inhibition of AURKB activity attenuates collagen deposition in vivo. Our in vitro data demonstrate that AURKB increases fibroproliferation and myofibroblast survival and inhibition of AURKB using barasertib attenuates fibroblast activation. To determine in vivo therapeutic effects of barasertib, TGFα mice were treated simultaneously with barasertib (40 mg/kg; QD) and Dox for four wks., a period that leads to lung fibrosis. The increase in lung weights and collagen deposition in the lung was attenuated in mice treated with barasertib versus vehicle-treated fibrosis controls (FIG. 11). These data establish that inhibiting AURKB phosphorylation is sufficient to attenuate pulmonary fibrosis in vivo. These data establish that inhibiting AURKB activity alters the progression of fibrosis. To test the efficacy of barasertib in inhibiting the established and ongoing pulmonary fibrosis, TGFα mice were treated with Dox for two weeks to induce pulmonary fibrosis and randomized into four groups (n=4/group), receiving either vehicle alone, low dose (25 mg/kg; QD) or high dose (50 mg/kg; QD) of barasertib or nintedanib (60 mg/kg; QD) for 5 days. TGFα mice on Dox for two weeks and treated with vehicle while on Dox for a week served as a control. The effect of barasertib on fibroproliferation was analyzed by immunostaining the lung sections with the cell proliferation marker Ki67; we observed a reduction in fibroproliferation in barasertib and nintedanib treated mice compared to vehicle treated TGFα mice (data not shown). TGFα mice treated with barasertib had a dose-dependent reduction in the expression of genes involved in fibroproliferation in the lungs (FIG. 12). We observed increases in the transcripts of pro-apoptotic genes in mice treated with barasertib (FIG. 13). Further, expression of ECM genes was attenuated in barasertib or nintedanib treated TGFα mice compared to vehicle treated control mice (FIG. 14).

The headings used in the disclosure are not meant to suggest that all disclosure relating to the heading is found within the section that starts with that heading. Disclosure for any subject may be found throughout the specification.

It is noted that terms like “preferably,” “commonly,” and “typically” are not used herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

As used in the disclosure, “a” or “an” means one or more than one, unless otherwise specified. As used in the claims, when used in conjunction with the word “comprising” the words “a” or “an” means one or more than one, unless otherwise specified. As used in the disclosure or claims, “another” means at least a second or more, unless otherwise specified. As used in the disclosure, the phrases “such as”, “for example”, and “e.g.” mean “for example, but not limited to” in that the list following the term (“such as”, “for example”, or “e.g.”) provides some examples but the list is not necessarily a fully inclusive list. The word “comprising” means that the items following the word “comprising” may include additional unrecited elements or steps; that is, “comprising” does not exclude additional unrecited steps or elements.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

Detailed descriptions of one or more embodiments are provided herein. It is to be understood, however, that the present invention may be embodied in various forms. Therefore, specific details disclosed herein (even if designated as preferred or advantageous) are not to be interpreted as limiting, but rather are to be used as an illustrative basis for the claims and as a representative basis for teaching one skilled in the art to employ the present invention in any appropriate manner. Indeed, various modifications of the invention in addition to those described herein will become apparent to those skilled in the art from the foregoing description and the accompanying figures. Such modifications are intended to fall within the scope of the appended claims.

What is claimed is:

1. A method for treating an animal for fibrosis, comprising one or more administrations of one or more compositions comprising one or more AURKB (Aurora kinase B) inhibitors, wherein the compositions may be the same or different if there is more than one administration.

2. The method of claim 1, wherein at least one of the one or more AURKB inhibitors is an AURKB antagonist, an AURKB partial antagonist, an AURKB inverse agonist, an AURKB partial inverse agonist, or a combination thereof.

3. The method of claim 1 or claim 2, wherein at least one of the one or more AURKB inhibitors further inhibits one or more of AURKA (Aurora kinase A), AURKC (Aurora kinase C), JAK2 (Janus kinase 2), JAK3 (Janus kinase 3), IGF-1R (Insulin-like growth factor 1 receptor), insulin receptor, MET (Hepatocyte growth factor receptor), ALK (Anaplastic lymphoma kinase), TRKA (Tropomyosin receptor kinase A), TRKB (Tropomyosin receptor kinase B), FLT3 (fms like tyrosine kinase 3), CDK1, (Cyclin-dependent kinase 1), CDK2 (Cyclin-dependent kinase 2), KDR (Kinase insert domain receptor), or a combination thereof.

4. The method of any of claims 1-3, wherein at least one of the one or more AURKB inhibitors further inhibits AURKA (Aurora kinase A), AURKC (Aurora kinase C), or both.

5. The method of any of claims 1-4, wherein at least one of the one or more AURKB inhibitors is AD6 (4-[(5-bromo-1,3-thiazol-2-yl)amino]-N-methyl-benzamide); AJI-100 (N4-(2-Chlorophenyl)-N2-(4-carbamoyl)-5-fluoropyrimidine-2,4-diamine); AJI-214 (N4-(phenyl)-N2-(4-carbamoyl)-5-fluoropyrimidine-2,4-diamine); AMG-900 (CAS number 945595-80-2; N-(4-(3-(2-aminopyrimidin-4-yl)pyridin-2-yloxy)phenyl)-4-(4-methylthiophen-2-yl)phthalazin-1-amine); AT9283 (CAS number 896466-04-9; 1-cyclopropyl-3-[(3Z)-3-[5-(morpholin-4-ylmethyl)benzimidazol-2-ylidene]-1,2-dihydropyrazol-4-yl]urea)); Aurora Kinase Inhibitor II (AI II) (CAS number 331770-21-9; N-[4-[(6,7-dimethoxy-4-quinazolinyl)amino]phenyl]benzamide); AZD1152 (CAS number 722543-31-9; 2-[ethyl-[3-[4-[[5-[2-(3-fluoroanilino)-2-oxoethyl]-1H-pyrazol-3-yl]amino]quinazolin-7-yl]oxypropyl]amino]ethyl dihydrogen phosphate); Barasertib (also known as AZD1152-HQPA or AZD2811) (CAS number 722544-51-6; 3-[[7-[3-[Ethyl(2-hydroxyethyl)amino]propoxy]-4-quinazolinyl]amino]-N-(3-fluorophenyl)-1H-pyrazole-5-acetamide); BI-811283 (4-((4-(((1R,2S)-2-(isopropylcarbamoyl)cyclopentyl)amino)-5-(trifluoromethyl)pyrimidin-2-yl)amino)-N-methyl-N-(1-methylpiperidin-4-yl)benzamide); BMS-754807 (CAS number 1001350-96-4; (2S)-1-[4-[(5-Cyclopropyl-1H-pyrazol-3-yl)amino]pyrrolo[2,1-f][1,2,4]triazin-2-yl]-N-(6-fluoro-3-pyridinyl)-2-methyl-2-Pyrrolidinecarboxamide); CCT129202 (CAS number 942947-93-5; 2-(4-(6-chloro-2-(4-(dimethylamino)phenyl)-3H-imidazo[4,5-b]pyridin-7-yl)piperazin-1-yl)-N-(thiazol-2-yl)acetamide); Chiauranib (CAS number 1256349-48-0; N-(2-aminophenyl)-6-((7-methoxyquinolin-4-yl)oxy)-1-naphthamide); CYC116 (CAS number 693228-63-6; 4-methyl-5-(2-(4-morpholinophenylamino)pyrimidin-4-yl)thiazol-2-amine); ENMD-2076 (CAS number 934353-76-1; (E)-N-(5-methyl-1H-pyrazol-3-yl)-6-(4-methylpiperazin-1-yl)-2-styrylpyrimidin-4-amine); GSK-1070916 (CAS number 942918-07-2; 3-(4-(4-(2-(3-((dimethylamino)methyl)phenyl)-1H-pyrrolo[2,3-b]pyridin-4-yl)-1-ethyl-1H-pyrazol-3-yl)phenyl)-1,1-dimethylurea); Hesperadin (CAS number 422513-13-1; N-[(3Z)-2-Oxo-3-[phenyl-[4-(piperidin-1-ylmethyl)anilino]methylidene]-1H-indol-5-yl]ethanesulfonamide); Ilorasertib (aka ABT-348; CAS number 1227939-82-3; 1-(4-(4-amino-7-(1-(2-hydroxyethyl)-1H-pyrazol-4-yl)thieno[3,2-c]pyridin-3-yl)phenyl)-3-(3-fluorophenyl)urea); JNJ-7706621 (CAS number 443797-96-4; 4-[5-amino-1-(2,6-difluoro-benzoyl)-1H-[1,2,4]triazol-3-ylamino]-benzenesulfonamide); KW-2449 (CAS number 1000669-72-6; (E)-(4-(2-(1H-indazol-3-yl)vinyl)phenyl)(piperazin-1-yl)methanone); KW-2450 (CAS number 904899-25-8; (E)-N-(2-(2-(1H-indazol-3-yl)vinyl)-5-((4-(2-hydroxyacetyl)piperazin-1-yl)methyl)phenyl)-3-methylthiophene-2-carboxamide 4-methylbenzenesulfonate) or its tosylate salt; MK-6592 (aka VX667; (S)-(5-chloro-2-fluorophenyl)(3-(4-(3-cyclopropyl-3-fluoroazetidin-1-yl)-6-(3-methyl-1H-pyrazol-5-ylamino)pyrimidin-2-yloxy)pyrrolidin-1-yl)methanone); MLN8054 (CAS number 869363-13-3; 4-((9-chloro-7-(2,6-difluorophenyl)-5H-benzo[c]pyrimido[4,5-e]azepin-2-yl)amino)benzoic acid); MLN8237 (CAS number 1028486-01-2; 4-{[9-Chloro-7-(2-fluoro-6-methoxyphenyl)-5H-pyrimido[5,4-d][2]benzazepin-2-yl]amino}-2-methoxybenzoic acid); PF-03814735 (CAS number 942487-16-3; N-[2-[(1S,4R)-6-[[4-(Cyclobutylamino)-5-(trifluoromethyl)-2-pyrimidinyl]amino]-1,2,3,4-tetrahydronaphthalen-1,4-imin-9-yl]-2-oxoethyl]-acetamide) or its mesylate salt; PHA-680632 (CAS number 398493-79-3; N-(2,6-diethylphenyl)-3-[[4-(4-methylpiperazin-1-yl)benzoyl]amino]-4,6-dihydro-1H-pyrrolo[3,4-c]pyrazole-5-carboxamide); PHA-739358 (aka Danusertib; CAS number 827318-97-8; 4-(4-methyl-1-piperazinyl)-N-[1,4,5,6-tetrahydro-5-[(2R)-2-methoxy-2-phenylacetyl]pyrrolo[3,4-c]pyrazol-3-yl]-benzamide); SNS314 (CAS number 1146618-41-8; 1-(3-chlorophenyl)-3-[5-[2-(thieno[3,2-d]pyrimidin-4-ylamino)ethyl]-1,3-thiazol-2-yl]urea) or its mesylate salt; SU6668 (CAS number 252916-29-3; 5-[1,2-Dihydro-2-oxo-3H-indol-3-ylidene)methyl]-2,4-dimethyl-1H-pyrrole-3-propanoic acid); TAK-901 (CAS number 934541-31-8; 5-(3-(ethylsulfonyl)phenyl)-3,8-dimethyl-N-(1-methylpiperidin-4-yl)-9H-pyrido[2,3-b]indole-7-carboxamide); TX47 (3,3′-((1H-indole-2,3-diyl)bis(methylene))bis(1H-indole)); TY-011 (9-(2-chloro-phenyl)-6-ethyl-1-methyl-2,4-dihydro-2,3,4,7,10-pentaaza-benzo[f]azulene); VX-680 (aka Tozasertib; CAS number 639089-54-6; N-[4-[4-(4-Methylpiperazin-1-yl)-6-[(5-methyl-1H-pyrazol-3-yl)amino]pyrimidin-2-yl]sulfanylphenyl]cyclopropanecarboxamide); or ZM447439 (CAS number 331771-20-1; N-[4-[[6-methoxy-7-[3-(4-morpholinyl)propoxy]-4-quinazolinyl]amino]phenyl]-benzamide); or a salt, ester, or solvate of any of the aforementioned.

6. The method of any of claims 1-5, wherein at least one of the one or more AURKB inhibitors is AMG-900; AT-9283; AZD1152; Barasertib; BI-811283; Chiauranib; CYC-116; ENMD-2076; GSK-1070916; Ilorasertib; KW-2449; MK-6592; PF-03814735 or its mesylate salt; PHA-739358 (aka Danusertib); TAK-901; SNS-314 or its mesylate salt; or VX-680 (aka Tozasertib); or a salt, ester, or solvate of any of the aforementioned.

7. The method of any of claims 1-5, wherein at least one of the one or more AURKB inhibitors is AD6; AJI-100; AJI-214; AT9283; Aurora Kinase Inhibitor II (AI II); AZD1152; Barasertib; BMS-754807; CYC116; hesperadin; JNJ-7706621; KW-2450 or its tosylate salt; PF-03814735 or its mesylate salt; TX47; TY-011; or ZM447439; or a salt, ester, or solvate of any of the aforementioned.

8. The method of any of claims 1-5, wherein at least one of the one or more AURKB inhibitors is AZD1152; BRD-7880; Barasertib; GSK1070916; or TAK-901; or a salt, ester, or solvate of any of the aforementioned.

9. The method of any of claims 1-8, wherein at least one of the one or more AURKB inhibitors is AZD1152 or Barasertib, or a salt, ester, or solvate of Barasertib or of AZD1152.

10. The method of any of claims 1-9, wherein the amount of at least one of the one or more AURKB inhibitors is from about 0.0001% (by weight total composition) to about 99%.

11. The method of any of claims 1-10, wherein at least one of the one or more compositions further comprises a formulary ingredient.

12. The method of any of claims 1-11, wherein at least one of the one or more compositions is a pharmaceutical composition.

13. The method of any of claims 1-12, wherein at least one of the one or more administrations comprises a parenteral administration, a mucosal administration, intravenous administration, a depot injection, a subcutaneous administration, a topical administration, an intradermal administration, an oral administration, a sublingual administration, an intratracheal administration, an intranasal administration, an intramuscular administration, an aerosol administration, a nebulizer administration, a pressurized metered-dose inhaler (pMDI) administration, an inhaler administration, or a dry powder inhaler (DPI) administration.

14. The method of any of claims 1-13, wherein at least one of the one or more administrations comprises an intratracheal administration, an intranasal administration, an aerosol administration, a nebulizer administration, a pressurized metered-dose inhaler (pMDI) administration, an inhaler administration, or a dry powder inhaler (DPI) administration.

15. The method of any of claims 1-14, wherein if there is more than one administration at least one composition used for at least one administration is different from the composition of at least one other administration.

16. The method of any of claims 1-15, wherein at least one of the AURKB inhibitors of at least one of the one or more compositions is administered to the animal in an amount of from about 0.005 mg/kg animal body weight to about 100 mg/kg animal body weight.

17. The method of any of claims 1-16, wherein the animal is a human, a rodent, or a primate.

18. The method of any of claims 1-17, wherein the animal is in need of treatment of fibrosis.

19. The method of any of claims 1-18, wherein the method is for treating lung fibrosis, skin fibrosis, kidney fibrosis, liver fibrosis, gastrointestinal fibrosis, heart fibrosis, brain fibrosis, arterial stiffness, arthrofibrosis, crohn's disease, dupuytren's contracture, keloid, mediastinal fibrosis, myelofibrosis, peyronie's disease, nephrogenic systemic fibrosis, progressive massive fibrosis, retroperitoneal fibrosis, scleroderma/systemic sclerosis, adhesive capsulitis, or other organ fibrosis.

20. The method of any of claims 1-19, wherein the method is for treating lung fibrosis, pulmonary fibrosis, cystic fibrosis, idiopathic pulmonary fibrosis (IPF), radiation-induced lung injury, skin fibrosis, kidney fibrosis, liver fibrosis, cirrhosis, heart fibrosis, atrial fibrosis, endomyocardial fibrosis, myocardial infarction, gastrointestinal fibrosis, fibrosis of the gastrointestinal tract, fibrosis associated with gastrointestinal inflammation, fibrosis associated with inflammatory bowel disease, fibrosis associated with ulcerative colitis, fibrosis associated with Crohn's disease, intestine fibrosis, small intestine fibrosis, ilium fibrosis, cecum fibrosis, or colon fibrosis.

21. The method of any of claims 1-20, wherein the method is for treating lung fibrosis, pulmonary fibrosis, cystic fibrosis, idiopathic pulmonary fibrosis (IPF), or radiation-induced lung injury.

22. The method of any of claims 1-21, wherein the method is for treating gastrointestinal fibrosis, fibrosis of the gastrointestinal tract, fibrosis associated with gastrointestinal inflammation, fibrosis associated with inflammatory bowel disease, fibrosis associated with ulcerative colitis, fibrosis associated with Crohn's disease, intestine fibrosis, small intestine fibrosis, ilium fibrosis, cecum fibrosis, or colon fibrosis.

23. The method of any of claims 1-22, wherein the method is for treating liver fibrosis, kidney fibrosis, or skin fibrosis.

24. The method of any of claims 1-23, wherein the method further comprises one or more other fibrosis treatments.

25. The method of any of claims 1-24, wherein the method further comprises one or more other fibrosis treatments and the other fibrosis treatment comprises administering one or more of an antibiotic, an anti-inflammatory drug, a mucus thinner, or an antifibrotic medication.

26. The method of any of claims 1-25, wherein the method further comprises one or more other fibrosis treatments and the other fibrosis treatment comprises administering one or more non-drug respiratory therapies.

27. A method for treating a human for lung fibrosis, pulmonary fibrosis, or idiopathic pulmonary fibrosis (IPF), comprising
administering one or more compositions comprising barasertib or AZD1152.

28. The method of claim 27, wherein at least one of the one or more compositions comprises AZD1152.

29. A method for treating a human for idiopathic pulmonary fibrosis (IPF), comprising
administering one or more compositions comprising barasertib or AZD1152, wherein the administering is by a pressurized metered-dose inhaler (pMDI) administration, an inhaler administration, or a dry powder inhaler (DPI) administration.

30. The method of claim 29, wherein at least one of the one or more compositions comprises barasertib.
